(19) United States
(12) Patent Application Publication
(10) Pub. No.: US 2021/0308335 A1
(43) Pub. Date: Oct. 7, 2021

(54) Fibrous 3-Dimensional Scaffold Via Electrospinning For Tissue Regeneration and Method For Preparing the Same

(71) Applicant: EWHA UNIVERSITY -INDUSTRY COLLABORATION FOUNDATION, Seoul (KR)

(72) Inventors: Seung Jin Lee, Seoul (KR); Sol Han, Seoul (KR); In Kyong Shim, Seoul (KR); Young Il Yang, Busan (KR); Yang Soo Jang, Seoul (KR); Mi Ra Jung, Seoul (KR); Hye Jin Chung, Seoul (KR)

(73) Assignee: EWHA UNIVERSITY -INDUSTRY COLLABORATION FOUNDATION, Seoul (KR)

(21) Appl. No.: 14/047,948

(22) Filed: Oct. 7, 2013

Related U.S. Application Data
(63) Continuation-in-part of application No. 12/064,801, filed on Feb. 25, 2008, now abandoned.
Application No. 12/064,801 is a continuation-in-part of application No. PCT/KR2013/005094, filed on Jun. 10, 2013.

(30) Foreign Application Priority Data
Aug. 26, 2005 (KR) 10-2005-0078640
Jun. 8, 2012 (KR) 10-2012-0061818

(51) Int. Cl. A61L 27/56; D01D 5/00; D01D 7/00; D01D 11/00; D01F 6/62; A61L 27/36; C12M 1/12

(52) U.S. Cl. CPC A61L 27/56; D01D 5/0038; D01D 7/00; D01D 11/00; D01F 6/625; D10B 2401/08; C12M 25/14; A61L 2430/20; A61L 2430/30; D10B 2401/10; D10B 2401/12; A61L 27/3625

(57) ABSTRACT

The present invention relates to a fibrous 3-dimensional porous scaffold obtained by electro-spinning for tissue regeneration and a method for preparing the same.

### TECHNICAL FIELD

The present invention relates to a fibrous 3-dimensional porous scaffold obtained via electro-spinning for tissue regeneration and a method for preparing the same.

### BACKGROUND ART

Tissue regeneration is induced by supplying cells or drug-loaded matrix when tissues or organs lose the ability to perform their functions or are damaged. Currently, a scaffold for tissue regeneration has to be physically stable in the implanted site, has to be physiologically active to control regeneration efficacy, has to be easily degradable in vivo after generating new tissues and must not produce degradation products with toxicity.

Conventional scaffolds for tissue regeneration have been produced by using polymers having a certain strength and form, for example, sponge type, fibrous matrix and gel type cell culture scaffolds have been used.

The conventional fibrous matrix scaffold has open cellular pores and the pore size is of a large enough size that cells are easily adherable and can proliferate. However, the fibrous matrix scaffold is not commonly used today as its disadvantages have been confirmed as follows: a scaffold composed of a natural polymer has such a poor strength in the water phase that it might contract or be destroyed, thereby losing its original form, and even a synthetic polymer scaffold cannot secure a room with its fibrous structure alone, so that it ends up as a membrane shaped 2-dimensional structure rather than 3-dimensional structure. Having a 3-dimensional structure is very important for tissue regeneration and activity. So, scaffolds having only a 2-dimensional structure are limited in application since it is very difficult for these scaffolds to envelop a medicine and regulate its release or to employ a natural polymer with high physiological activity.

The preparation method for sponge type scaffolds has been generally accepted for the preparation of conventional scaffolds for tissue generation, for example, particle leaching, emulsion freeze-drying, high pressure gas expansion and phase separation, etc.

The particle leaching technique is that wherein particles which are insoluble in bio-degradable polymer with an organic solvent such as salt are mixed with a casting, the solvent is evaporated and then the salt particles are eliminated by elution in water. According to this method, a porous structure with cellular pores of different sizes and at various porosities can be obtained by regulating the size of the salt particle and the mixing ratio. However, there is a problem in this method that the remaining salts or the rough surface cause cell damage (Mikos et al., Biomaterials, 14: 323-330, 1993; Mikos et al., Polymer, 35: 1068-1077, 1994).

Emulsion freeze-drying is the method wherein the emulsion of a polymer with organic solvent and water is freeze-dried to eliminate the residual solvents. In the meantime, the high pressure gas expansion method does not use any organic solvent. According to this method, a bio-degradable polymer is introduced into a mold and pressure is applied thereto to prepare the pellet. Then, high pressure carbon dioxide is injected into the biodegradable polymer at a proper temperature and then the pressure is reduced to release carbon dioxide in the mold to form cellular pores. However, the above methods are also limited in producing open cellular pores (Wang et al., Polymer, 36: 837-842, 1995; Mooney et al., Biomaterials, 17: 1417-1422, 1996).

Another attempt has recently been made to prepare a porous scaffold based on phase separation. Particularly, a sublimable substance or another solvent having different solubility is added to a polymer organic solvent and then phase separation of the solution is performed by sublimation or temperature change. However, this method has also a difficult problem regarding the cell culture because the size of the produced pore is too small (Lo et al., Tissue Eng. 1: 15-28, 1995; Lo et al., J. Biomed. Master. Res. 30: 475-484, 1996; Hugens et al., J. Biomed. Master. Res., 30: 449-461, 1996).

The above-mentioned methods are to prepare a 3-dimensional polymer scaffold which is capable of inducing cell adhesion and differentiation, but practically using a biodegradable polymer for the production of a 3-dimensional scaffold for tissue regeneration still has a lot of problems to be overcome.

A polymer scaffold prepared by using electrospinning has been evaluated, but the results confirmed that it ends up as a 2-dimensional membrane structure, which means it is very difficult to use this scaffold as a 3-dimensional structured implantation material for successful cell adhesion (Yang et al., J. Biomater. Sci. Polymer Edn., 5: 1483-1479, 2004; Yang et al., Biomaterials, 26: 2603-2610, 2005).

An extracellular matrix in vivo has a network-structure composed of basic materials such as glycosaminoglycan and collagen nanofiber, in which cells are adhered and proliferated to form tissues.

With this background, the inventors developed a 3D scaffold in which separable biodegradable fibers with a certain thickness and porosity are entangled, instead of the conventional 2D fibrous membrane, and carried out examination for the ability of the scaffold to regenerate tissues after loading cells or drugs thereto, finding that the scaffold can be directly applied to tissues without operational difficulty, and can alternatively expand or shrink in response to the expansion or contraction of the tissue it was applied to, as well as the growth and the migration of the cells. In addition, when the electrospun microfibrous mat is mechanically expanded to create a 3D scaffold of the present invention, optimal electrospining conditions which can form meshes in which the electrospun fibers are separably entangled with each other was investigated, and the prepared scaffold has pore sizes and porosity large enough to effectively promote the growth of cells loaded thereto, thereby completing the present invention.

### DISCLOSURE OF INVENTION

### Technical Problem

It is an object of the present invention to provide a fluffy 3-dimensional (3D) porous scaffold comprising biodegradable polymer fibers, wherein the biodegradable polymer fibers in the scaffold are separably entangled with each other to form a 3D network structure.

It is another object of the present invention to provide a method for preparing the scaffold, comprising: (i) preparing a spinning solution by dissolving biodegradable polymers in an organic solvent; (ii) spinning the spinning solution by using an electro-spinner and volatilizing the organic solvent at the same time to form a microfibrous mat comprising biodegradable polymer fibers, which are separably entangled with each other in a network structure; and (iii) expanding the microfibrous mat mechanically to form the fluffy 3D porous scaffold.

It is another object of the present invention to provide a implantation material for tissue regeneration comprising the scaffold.

### Technical Solution

One aspect, the present invention provides a fluffy 3-dimensional (3D) porous scaffold comprising biodegradable polymer fibers, wherein the biodegradable polymer fibers in the scaffold are separably entangled with each other to form a 3D network structure.

Another aspect, the present invention provides a method for preparing the scaffold, comprising: (i) preparing a spinning solution by dissolving biodegradable polymers in an organic solvent; (ii) spinning the spinning solution by using an electro-spinner and volatilizing the organic solvent at the same time to form a microfibrous mat comprising biodegradable polymer fibers, which are separably entangled with each other in a network structure; and (iii) expanding the microfibrous mat mechanically to form the fluffy 3D porous scaffold.

Another aspect, the present invention provides a implantation material for tissue regeneration comprising the scaffold.

Hereinafter, the present invention is described in detail.

Conventional scaffolds for tissue regeneration are likely to tear upon transplantation into tissues or are unlikely to be brought into close contact with curved surfaces of the organs since they are formed into monolayer membrane structures with devoid of pore, or into thin layer structures lacking flexibility with smaller pore sizes than the cellular diameter. In addition, they are of low porosity, and do not readily allow for deep cell infiltration thereinto.

In light of this background, a fluffy 3-dimensional scaffold in which separable, biodegradable fibers are entangled with each other was developed by the present invention. Having these structural and morphological features, the scaffold of the present invention is allowed to swell up or to expand the pores between entangled fibers when one or more physical forces are applied thereto in opposite directions.

In one embodiment of the present invention, when one or more physical forces were applied in opposite directions to the fluffy scaffold in which separable, biodegradable, electrospun fibers are entangled, the scaffold was observed to increase in total volume and expand the pores between the fibers to the extent that the thickness thereof increased to 1 cm or greater. Thus, the scaffold of the present invention can be alternatively expanded or shrunk depending on environmental conditions (FIGS. 5 and 15 to 17).

Even when implanted into the heart, the scaffold for tissue regeneration of the present invention can elastically shrink or expand in response to the systole and diastole cycle of the heart, with the retention of mechanical strength sufficient to endure the compression attributable to the heart beating. In brief, the scaffold of the present invention is flexible enough to control its volume or thickness depending on the environment where it is implanted.

In addition, since it has a fluffy structure such that the application of a physical force thereto increases the total volume and expands pores between entangled fibers without breaking fibers, the scaffold for tissue regeneration of the present invention assures a space in which cells seeded in the scaffold proliferate or move to an ischemic zone without being stifled, and guarantees oxygen permeability through the expanded pores.

In one embodiment of the present invention, when implanted to a damaged bone tissue of an animal, the osteoblast-loaded scaffold of the present invention exhibited superior viability for osteoblasts loaded into the scaffold, and bone regeneration, compared to nanofiberous membranes or as-spun microfibrous mats. In animals where a scaffold for tissue regeneration containing cardiac stem cells and/or a growth factor was transplanted to the myocardium, the stem cells were alive for a prolonged period of time in the scaffold, undergoing proliferation, migration into cardiac muscles and differentiation into cardiomyocytes, with the consequent attainment of effects including angiogenesis in ischemic and recipient regions, thickening the anterior wall of the left ventricle, and myocardial regeneration.

According to one embodiment of the present invention, the 3D porous scaffold of the present invention can be used for stem cell implantation. The cells were discovered to survive and keep going at much higher rates when they were implanted to a damaged site with the aid of the 3D porous scaffold of the present invention than alone or by using fibrin gel.

In a further embodiment of the present invention, the 3D porous scaffold of the present invention was observed to allow for the stable loading of a gene thereinto. Thus, when loaded with a gene, the scaffold could release the genes locally in a controlled manner.

Consequently, the scaffold for tissue regeneration of the present invention can be attached directly to a target tissue, and enables stem cells to be engrafted into and maintained in a damaged site at a high rate, thereby overcoming the problem with conventional patches that after implantation to the heart, cells survive only at a low rate and thus have little influence on the regeneration of myocardial tissues. Hence, the scaffold of the present invention has viable applications for angiogenesis and tissue regeneration in damaged tissue sites.

The scaffold of the present invention is a fluffy 3D porous electrospun scaffold for tissue regeneration, comprising polymers which are formed in a 3-dimensional network structure.

In the 3D scaffold, the fibers in a network structure are separated without two-dimensionally clinging to each other, and form a fluffy, porous structure in which fibers ar entangled to form the pores. The conventional 2D matrix has limitation because most of the fibers fuse at the contact points, which leads to the fabrication of only dense membrane shapes and, typically smaller pore sizes than the cellular diameter. On the other hand, the 3D porous scaffold of the present invention, in which the fibers are entangled with each other, has no or little interfiber bonding, and thus the 3D scaffold of the present invention can be mechanically expanded until it has high porosity and large pore size, without breaking fibers.

In detail, separable, biodegradable fibers in an entangled fluffy network structure can alternatively expand or shrink in one-, two- or three-dimensional patterns in response to the swelling or contraction of the tissues to which the fibers are attached. By way of example, when implanted to a cardiac tissue, the scaffold of the present invention can alternatively expand or shrink as the heart beats or according to the contour of the cardiac tissue. In addition, the total volume of the scaffold or the pore size between entangled fibers may be adjusted as the cells introduced into the 3D scaffold proliferate.

Conventional 3D scaffolds in a spongy form are poor in adhesion because they are likely to recover after attachment to a damaged tissue. In contrast, a patch based on the porous 3D scaffold of the present invention is highly adhesive to a damaged site and can be attached directly to an organ even if it has a curved contour or surface.

In consideration of oxygen permeability, the space for cell proliferation and migration, and tissue specificity, the thickness of the scaffold for tissue regeneration according to the present invention may be determined appropriately. In addition, the scaffold may have a size large enough to protect an affected zone.

The scaffold preferably ranges in thickness from 50 μm to 1.5 cm, but is not limited thereto. For use in the heart, the patch might be preferably 0.1 to 3.5 mm thick, and more preferably 0.1 to 3 mm thick. The thickness of the patch may be preferably adjusted by applying one or more physical forces in opposite directions to the patch to increase the total volume. After being spun from a spinner, the scaffold is preferably expanded in total volume by twice or more by applying a physical force thereto. More preferably, the total volume is 2 to 15 times increased.

Since the scaffold might have dense local sites into which the cells cannot penetrate when the scaffold swells too thickly by physical expansion, it is preferred that the thickness of the patch be adjusted according to the properties of the tissues in the implant area.

The size and distribution of pores in the scaffold is a very important factor in determining cell growth in the scaffold. The pores should be inter-connected sufficiently for nutrients to uniformly penetrate into the scaffold, thereby allowing for good cell growth across the patch. Preferably, the scaffold has a pore size ranging from 50 μm to 400 μm, and most preferably a pore size of 100 μm. The pore sizes may vary according to the types of cells or drugs applied to the scaffold of the present invention. These pore sizes are suitable for facilitating the penetration of cells and the passage of nutrients and waste matter, helping the implanted cells grow, and promoting angiogenesis after implantation. To increase the pore size, one or more physical forces may be applied in opposite directions to the patch. In addition, the scaffold of the present invention may have a 3D structure with a porosity of 30 to 99% and preferably 50 to 99% after the volumetric expansion of pores, thus improving the growth of cells for tissue regeneration.

No particular limitations are imposed on the shape and area of the scaffold. Its size is large enough to cover a target site.

In addition, the 3D scaffold of the present invention may have an orientation.

Since the cardiac muscle exhibits orientation and exerts pressures, the patch to be applied to a cardiac tissue should be in an oriented network structure.

In this regard, the scaffold is electrospun on a cylindrical drum collector, instead of a typical stainless steel plate collector, at a rotation speed of 1,000 rpm or higher so as to prepare a patch in an oriented network structure.

As used herein, the term “orientation” refers to pertaining to the lengthwise distribution of the fibers which is not random, but provided with directionality, as shown in FIG. 18.

In one embodiment of the present invention, an oriented porous, fibrous 3D scaffold was prepared and loaded with stem cells and optionally a growth factor, so that the cells proliferated well, with orientation in a pattern similar to that of myocardiocytes with orientation. In addition, the growth factor was observed to be uniformly distributed across the fibers, showing a sustained-release modality.

The scaffold for tissue regeneration in accordance with the present invention is applicable directly to most of the organs which require cell delivery thereto because of having been damaged. So long as it allows for the application of a scaffold thereto, any organ is available in the present invention. Particularly, the scaffold of the present invention can be used to effectively regenerate tissues after being applied to heart, liver, skin, bone, nerve, or pancreas when they are damaged, but the application is not limited thereto.

By way of example, when the patch loaded with cardiac stem cells is attached to heart, the cardiac stem cells differentiate into myocardiocytes and the implanted cells migrate into neighbor tissues, e.g., damaged cardiac tissues where the myocardial infarction occurred, so as to regenerate the damaged tissue.

In the scaffold for tissue regeneration according to the present invention, the cells to be loaded to the scaffold are preferably cells having differentiation potential (e.g., stem cells).

As adult stem cells, stem cells derived from adiposes, umbilical cords, or bone marrow may be useful. For use in regenerating damaged cardiac tissues, cardiac stem cells are preferable which can effectively differentiate into myocardiocytes with the concomitant regeneration of the damaged cardiac tissue, and can migrate into cardiac tissues. In one embodiment of the present invention, cardiac stem cells of mice were employed. However, stem cells taken from all mammals including humans, rats, monkeys, etc. can be loaded to the scaffold of the present invention. When the scaffold loaded with stem cells was transplanted to a damaged tissue, the stem cells were observed to differentiate into the cells responsible for the tissue. Particularly, cardiac stem cells can be promoted to differentiate using a heart-specific marker. Examples of the marker include, but are not limited to, MHC (myosin heavy chain), cTnI (cardiac troponin I), cTnT (cardiac troponin T), α-cardiac actin, α-actinin, and MLC2 (myosin light chain). The phenotype of these cells can be evaluated as rhythmical contraction, and by the expression of markers relevant to the heart.

For use in the rapid recovery of a damaged tissue, in addition, the scaffold for tissue regeneration according to the present invention can be loaded with a drug. The drug may be a gene involved in tissue regeneration, a gene for improving the function of stem cells, or a regenerating protein.

Preferably, the regenerating protein is one or more selected from the group consisting of, but not limited to, vascular endothelial growth factor-A (VEGF-A), VEGF-B, VEGF-C, VEGF-D, VEGF-E, neuropilin, fibroblast growth factor-1 (FGF-1), FGF-2 (bFGF), FGF-3, FGF-4, FGF-5, FGF-6, angiopoietin-1, angiopoietin-2, erythropoietin, BMP-2, BMP-4, BMP-7, TGF-beta, IGF-1, osteopontin, pleiotropin, activin, and endothelin-1. Vascular endothelial growth factors may be used independently or in combination. When used in combination, vascular endothelial growth factors act in synergy, overwhelming the sum of individual effects.

In one embodiment of the present invention, after the scaffold was loaded with osteoblasts, cell growth and activity were examined. As a result, activity and growth of the cells loaded into the scaffold of the present invention were excellent when compared to that of 2D scaffold. In addition, after osteoblast-loaded scaffold of the present invention was implanted to bone defects, the healing of bone defects were induced. Also, in one embodiment of the present invention, after the scaffold was loaded with VEGF as a drug, its release profile was examined. The drug was found to be released slowly with time. In addition, when implanted to damaged tissue, the scaffold loaded with stem cells plus a vascular endothelial stem cell brought about higher effects in tissue regeneration than that loaded with stem cells alone.

Capable of drug delivery, the scaffold of the present invention brings about a synergic effect on tissue regeneration. Particularly, the scaffold releases a drug in a sustained manner over the period of regeneration, further improving tissue regeneration effects.

Cells or stem cells may be loaded to the surface and/or inside of the scaffold.

The 3D scaffold for tissue regeneration of the present invention is preferably formed from a synthetic polymer which is biodegradable, preferably hydrolytically degradable, in terms of processability, sterilizability, and contamination resistance. Particularly, the scaffold is formed from hydrolysable polymers of α- and β-hydroxycarboxylic acid.

The fibrous porous scaffold of the present invention contains a bio-degradable polymer composed of one or more natural polymers selected from a group consisting of chitosan, chitin, alginic acid, collagen, gelatin and hyaluronic acid and/or a biodegradable polymer composed of a representative bio-degradable aliphatic polyester selected from a group consisting of polylactic acid (PLA), polyglycolic acid (PGA), poly(D,L-lactide-co-glycolide) (PLGA), poly(caprolactone), diol/diacid aliphatic polyester and polyester-amide/polyester-urethane and one or more synthetic polymers selected from a group consisting of poly(valerolactone), poly(hydroxyl butyrate) and poly(hydroxyl valerate).

The synthetic polymer may be low molecular PLA or high molecular PLA. Further, the synthetic polymer may have 0.5 dL/g to 7 dL/g of IV (inherent viscosity), but is not limited thereto. In addition, the synthetic polymer PLA is preferably PLLA.

The fibrous porous scaffold of the present invention has a size between nanofiber and microfiber, preferably 1-15 μm in diameter, and a regular form and strength under a proper pressure to help 3-dimensional tissue regeneration and at the same time to provide a large surface area for cell adhesion, so that it can be effectively used for adhesion and proliferation of such cells as myocardiocytes, endothelial cells, skin cells and osteocytes. Therefore, polymers in the spinning solution may be adjusted to form a fiber having the above diameter provided. Considering viscosity of polymers or spinning condition, the concentration of polymers in the spinning solution can be adjusted to form scaffold in which fibers have little or no interfiber bonding.

In addition, the scaffold of the invention can be simply prepared by using electrospinning without waste of polymers or drugs, so it can be more efficient than any other method.

The fibrous porous scaffold of the present invention can include not only a polymer but also a synthetic low molecular compound.

As another aspect, the present invention provides a method for preparing the scaffold.

Particularly, the present invention provides a method for preparing the scaffold, comprising: (i) preparing a spinning solution by dissolving biodegradable polymers in an organic solvent; (ii) spinning the spinning solution by using an electro-spinner and volatilizing the organic solvent at the same time to form a microfibrous mat comprising biodegradable polymer fibers, which are separably entangled with each other in a network structure; and (iii) expanding the microfibrous mat mechanically to form the fluffy 3D porous electrospun scaffold.

In the above step (i), to prepare the spinning solution which can prepare polymer fibers which have viscosity and diameter suitable for 3D scaffold of the present invention, a biodegradable polymer is dissolved in an organic solvent.

Any volatile organic solvent having a low boiling point can be used as an organic solvent for the invention to dissolve the synthetic polymer above and particularly chloroform, dichloromethane, dimethylformamide, dioxane, acetone, tetrahydrofurane, trifluoroethane, 1,1,1, 3,3, 3,-hexafluoroisopropylpropanol (HFIP), dichloromethane/HFIP or dicholoromethane/acetone are preferred and dichloromethane/acetone is more preferred but the solvent is not limited thereto. The proper organic solvent can be chosen depending on the situation.

In addition, the concentration of the biodegradable polymers can be adjusted to prepare the polymer fibers having 1 to 15 μm in diameter, which maintaining the structure of mechanically expanded scaffold of the present invention.

According to the present invention, the polymer solution drips on a collector by electrospinning and at this time the solvent is entirely volatilized. Because of electrostatic repulsive power, there is no direct contact between fiber and fiber, indicating that fibers are integrated separately. What is most important in this process is that all of the solvent has to be volatilized before the drip of the polymer solution on the collector, for which the boiling point of the solvent has to be very low and viscosity of the solvent has to be properly adjusted. Particularly, the preferable boiling point and viscosity of the solvent is 0-40° C. and 25-35 cps respectively. It is also important to maintain a proper temperature and humidity.

A polymer and a low molecular compound included in the fibrous 3-dimensional polymer scaffold may be dissolved in 4-20 weight % of an organic solvent to prepare a spinning solution.

According to the method for preparing the porous 3-dimensional scaffold of the invention, when temperature, humidity, viscosity of the solution and volatility of the solvent are optimized, fibers are not directly adhered and integrated separately, simply resulting in the microfibrous mat which can be mechanically expanded without breaking fibers.

In step (ii), a fiber is prepared by using the spinning solution with electro-spinner. According to the present invention, the polymer solution drips on a collector by electrospinning and at this time the solvent is entirely volatilized, resulting in the fibrous mat, in which the fibers are separably entangled with each other.

The spinning process by electro-spinner is described in detail hereinafter (see FIG. 1).

An electric field is formed between nozzle and collector by applying a certain current from voltage generator. The polymer solution filled in the spinning solution depository is spun on the collector by the force of the electric field and the pressure from syringe pump. At this time, voltage, flowing speed, the electric field distance between nozzle and collector, temperature and humidity are important factors affecting spinning. In particular, the concentration of the spinning solution affects the diameter of a fiber most significantly. So, all the conditions of the electro-spinner are optimized to prepare a fiber of the invention.

The conditions of the electro-spinner are as follows; spun distance: 10-20 cm, voltage: 10-20 kV, release speed: 0.050-0.150 ml/min, temperature: 15-25° C. and the internal diameter of the syringe: 0.5 to 1.2 mm, but not always limited thereto. The electro-spinner used in the present invention is DH High Voltage Generator (CPS-40KO3VIT, Chungpa EMT, Korea).

Further, a microfibrous mat which is oriented to one direction may be obtained using a cylindrical drum collector at a rotation speed of 1,000 rpm or higher, instead of a typical stainless steel collector.

The microfibrous mat prepared in the step (ii) is composed of the polymer fibers which are separable due to little binding between them and can be expanded mechanically with low fiber damage.

The above method can comprise (iii) expanding the electrospun microfibrous mat mechanically by applying one or more physical forces in opposite direction.

By applying the physical force thereto so as to increase the total volume and expand pores between fibers, the scaffold is preferably made fluffy with a thickness of 50 μm to 1.5 cm. In addition, the scaffold is preferably expanded about two times, more preferably two to 15 times of the initial volume after electrospinning.

In addition, the above method may further comprise (iv) introducing cells, a drug, a biologically active material, or a combination thereof into the scaffold of step (iii).

Herein, the cells useful in the present invention include stem cells allowing for tissue regeneration, and cells capable of differentiation. Among the drug available in the present invention are genes involved in tissue regeneration, genes accounting for improving the function of stem cells, and regenerating proteins.

A drug-loaded scaffold can be prepared by emulsifying an aqueous drug solution in a polymer solution, spinning the emulsion, and laminating the fibers. In the emulsification, the drug is dispersed in the biodegradable polymer solution. In this regard, the drug can be uniformly dispersed in a therapeutically effective amount in a biodegradable polymer solution using a surfactant well known in the art, such as span 80, to form a water-in-oil type emulsion.

When the drug-loaded scaffold is transplanted to damaged tissue and exposed to an aqueous environment, the drug, such as a protein, is released. At an early stage of release, the drug is diffused. Once the scaffold is degraded or collapses, the release of the drug from the cracks is increased. The release speed and profile can be controlled depending on various factors including the concentration of the protein, the ratio of oil/water, a site to which the scaffold is applied, etc.

As another aspect, the present invention provides the implantation material comprising the scaffold.

The scaffold and the types of cells applied thereto are same as the described above.

### BRIEF DESCRIPTION OF DRAWINGS

FIG. 1 is a schematic diagram illustrating the spinning using an electro-spinner.

FIG. 2 is a photomicrograph (×500) of fiber prepared under the conditions of double electric field length: 20 cm, voltage: 10 V, release rate: 0.060 ml/min., and inner diameter of needle: 1.2 mm.

FIG. 3 is a photomicrograph (×3500) of fiber prepared under the conditions of double electric field length: 20 cm, voltage: 10 V, release rate: 0.060 ml/min., and inner diameter of needle: 1.2 mm.

FIG. 4 is a photomicrograph (×2000) showing the surface of the fibrous porous scaffold prepared by electrospinning under the conditions of double electric field length: 20 cm, voltage: 10 V, release rate: 0.060 ml/min., and inner diameter of needle: 1.2 mm.

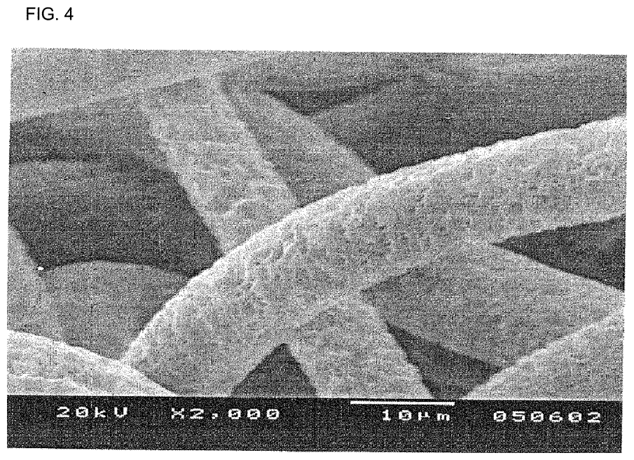

FIGS. 5A-5C is a photographic image (left) and scanning electron microscope image (right) of electrospun fibrous scaffolds. (a) Nanofibrous membranes, (b) microfibrous mats, and (c) 3D microfibrous scaffolds after mechanical expansion.

FIGS. 6A-6F show is a scanning electron microscope image of PLLA electrospun. Effect of PLLA concentration on the structure of the electrospun fibers at an onset voltage: (a) 2% w/v and 14 kV; (b) 4% w/v and 10 kV; (c) 6% w/v and 10 kV; (d) 8 wt % and 10 kV; (e) 9% w/v and 11 kV; and (f) 10% w/v and 18 kV.

FIGS. 7A-7C show is a scanning electron micrograph of electrospun fibers in 8% PLLA solution. Solvent properties were studied at the same conditions: (a) MC/DMF (90:10 v/v); (b) MC/HFIP (90:10 v/v); and (c) MC/acetone (90:10 v/v). The fused points is indicated by an arrow (↑).

FIG. 8 is a representative plot of cumulative incremental intrusion volume versus pore diameter of electrospun fibrous scaffolds: (∘) nanofibrous membranes, (•) as-spun microfibrous mats and (Δ) 3D microfibrous scaffolds after mechanical expansion.

FIG. 9 is a photomicrograph (×2000) showing osteoblasts cultured for 7 days in low molecular scaffold.

FIG. 10 is a set of photomicrograph (×500) showing osteoblasts cultured for 14 days in low molecular scaffold.

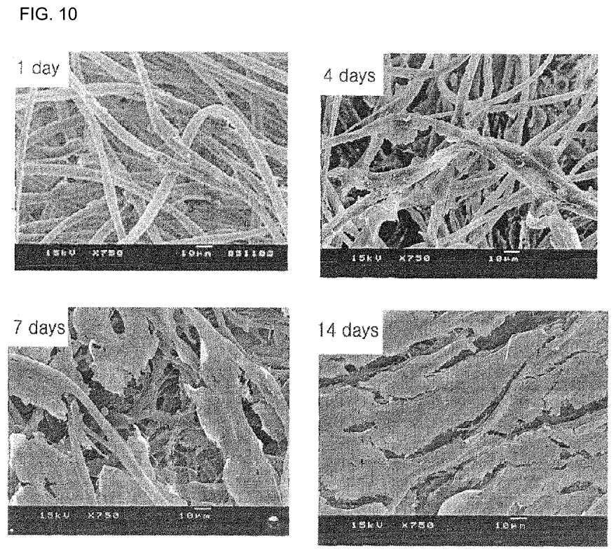

FIGS. 11A-11C show is SEM images of MC3T3-E1 cells cultured on electrospun fibrous scaffolds. (a) SEM images of cells on nanofibrous membranes; (b) on as-spun microfibrous mats; (c) on 3D microfibrous scaffolds after mechanical expansion. Original magnification of SEM images at 1 and 7 days are ×350. Arrow: attached MC3T3-E1 cells on the fibrous matrix; Arrowhead: PLLA fibers.

FIGS. 12A-12C show is SEM and H and E images of the cross-section of the cell/scaffold constructs after 14 days of incubation. (a) MC3T3-E1 cells on nanofibrous membranes; (b) on as-spun microfibrous mats; and (c) on 3D microfibrous scaffolds after mechanical expansion. Original magnification of SEM is ×150 and H and E is ×100 after 14 days, respectively. Arrow: attached MC3T3-E1 cells on the fibrous matrix; Arrowhead: PLLA fibers and asterisk: extracellular matrix. [Color figure can be viewed in the online issue which is available at wileyonlinelibrary.com.]

FIGS. 13A-13B show is a result of cell proliferation and ALPase activity of MC3T3 cells cultured on electrospun fibrous scaffolds. (a) Number of viable cells cultured on nanofibrous membranes (•), as-spun microfibrous mats (∘), or 3D microfibrous scaffolds after mechanical expansion (▾); (b) ALPase activity expressed by MC3T3-E1 cells on nanofibrous membranes (•), as-spun microfibrous mats (∘), or 3D microfibrous scaffolds after mechanical expansion (▾). The results are expressed as mean±S.E.M. (n ¼ 3). *, statistically significant compared to the cells cultured on nanofibrous membranes (p<0.05), **, statistically significant compared to the cells cultured on both nanofibrous membranes and as-spun microfibrous mats (p<0.05).

FIGS. 14A-14D show is microscopic observation of the H and E stained tissue sections of rabbit calvarial defects retrieved 2 weeks (a and b) and 4 weeks (c and d) after implantation; (a and c): no implantation as negative control; (b and d): treatment with electrospun 3D microfibrous scaffolds implant. Magnification of 20× for the full cross sections and 100× for the high magnification views of the defect margin area. The bone margin is indicated by a vertical arrow (↑). The loose connective tissues with fibroblast-like cells are indicated by an asterisk (*). New bone formation is indicated by an arrowhead (▴). Residual PLLA were indicated by circle (∘).

FIGS. 15A-15B show a PLLA fibrous porous 3D scaffold prepared in Example 7-(1) in an optical photograph (a) and in a differential scanning microphotograph (b).

FIG. 16 is a photograph showing that an electrospun fibrous mat can be expanded to a thickness of 1 cm or greater as physical force is applied thereto to increase inter-fibrous pores and the total volume.

FIG. 17 shows the reversible flexibility of the electrospun fibrous mat by gradually increasing the volume of the fibrous mat with the application of physical forces, in which the rightmost fibrous mat is completely different in the leftmost 3D scaffold, pore size between fibers, and porosity from the leftmost membranous fibrous mat.

FIG. 18 is a differential scanning microscopic image of a PLLA fibrous porous 3D scaffold prepared to have orientation.

FIGS. 19A-19B show shows a cardiac stem cell-loaded, non-oriented scaffold randomly spun according to Example 7-(1) (a) and a cardiac stem cell-loaded oriented scaffold prepared in Example 7-(2) (b) in differential scanning microscopic images.

FIGS. 20A-20B show_shows (a) a widthwise cross section and (b) a cross section of an oriented fibrous, porous 3D scaffold stained with H&E 48 hrs after the loading of cells thereto, in microscopic images.

FIG. 21 shows the distribution of a growth factor in the oriented 3D scaffold of Example 7-(3) as measured with a fluorescent dye.

FIG. 22 is a graph in which the cumulative release of VEGF from the oriented 3D scaffold of Example 7-(3) is plotted against time.

FIG. 23 is a graph showing counts of viable cells loaded to the scaffold of Example 7-(7) on day 1, day 4, day 7 and day 14.

FIG. 24 shows proliferation patterns of cells loaded to the 3D scaffold of Example 7-(1) (panels in row 2) and the oriented, 3D scaffold of Example 7-(2) (panels in row 4), illustrating that the cells in the oriented 3D scaffold proliferate in a morphologically similar pattern to that of myocardiocytes themselves.

FIGS. 25A-25B show cardiac stem cell-loaded scaffolds of Example 7-(8) stained with H&E, αSA, MHC, and TnT in microscopic images after incubation for 2 weeks in a growth medium (a) and a differentiation medium (b).

FIGS. 26A-26B show are microscopic images of normal tissues stained with H&E 4 days after cardiac stem cell-loaded scaffold of Example 7-(9) was transplanted onto the epicardium surface of the tissue.

FIGS. 27A-27D show an infarcted myocardium zone immediately after the cardiac stem cell-loaded scaffold of Example 7-(1) was transplanted (a), and the cardiac tissue 14 days after the cardiac stem cell-loaded scaffold of Example 7-(1) was transplanted (b), an infarcted myocardium zone immediately after the cardiac stem cell-free scaffold of Comparative Example 1 was transplanted (c), the cardiac tissue 14 days after the cardiac stem cell-free scaffold of Comparative Example 1 was transplanted (d).

FIGS. 28A-28B show tissues stained with H&E 14 days after the cardiac stem cell-loaded scaffold of Example 7-(1) was transplanted to the tissues (a) as described in Example 7-(10) and after the cardiac stem cell-free scaffold of Comparative Example 1 was transplanted to the tissues (b) as described in Example 7-(10).

FIGS. 29A-29C show fluorescence images of a cardiac tissue immunostained with αSA (a) and with DAPI (b) for nuclei after transplantation with the scaffold for 14 days according to Example 7-(10), and a merged image (c) thereof.

FIGS. 30A-30C show fluorescence images of a cardiac tissue immunostained with TnT (a) and with DAPI for nuclei (b) after transplantation with the scaffold for 14 days according to Example 7-(10), and a merged image (c) thereof.

FIGS. 31A-31C show fluorescence images of a tissue immunostained with SMA (a) and with DAPI for nuclei (b) after transplantation with the scaffold for 14 days according to Example 7-(10), and a merged image (c) thereof.

FIGS. 32A-32C show fluorescence images of a tissue immunostained with anti-CD34 antibody (a) and with DAPI for nuclei after transplantation with the scaffold for 14 days according to Example 7-(10), and a merged image (c) thereof.

FIGS. 33A-33C show low-magnification microscopic images of an entire scaffold and cardiac tissues transplanted with DiI-labeled cardiac stem cells in red fluorescence (a) and nucleus-stained with DAPI 14 days after transplantation according to Example 7-(10), and a merged image thereof (c).

FIGS. 34A-34C provide high-magnification microscopic images of cardiac tissues showing locations of the transplanted cardiac stem cells in the myocardium layer of the myocardium infarcted rats after transplantation thereinto, wherein the uppermost panels represent the attached scaffold while the more lower images are responsible for more inner myocardia with DiI-labeled cardiac stem cells in red fluorescence (a), DAPI-stained nuclei (b), and merged images thereof (c).

FIGS. 35A-35B show tissues stained with H&E 28 days after the cardiac stem cell-loaded scaffold of Example 7-(1) was transplanted to the tissues according to Example 7-(11) (a), and after the cardiac stem cell-free scaffold of Comparative Example 1 was transplanted to the tissues according to Example 7-(12).

FIG. 36 is a result of histological analysis of the tissues obtained 28 days after the scaffolds of Example 7-(1) and Comparative Example 1 were transplanted to the tissues according to Example 7-(11), showing their fibrotic area ratio (a) and LV wall thickness (b).

FIG. 37 shows the migration and penetration of cells into the 3D scaffolds which were swelled to a proper thickness through physical expansion, thereby allowing the cells to migrate and penetrate into the scaffolds (left panels) and which were swelled too much, thereby resulting in part of the scaffold for the cells not to migrate and penetrate into the scaffolds (white, right panel).

FIG. 38 shows the effect of the scaffold on myocardial tissue regeneration after the scaffold alone, the scaffold loaded with a growth factor, and the scaffold loaded with both a growth factor and cardiac stem cells were transplanted into myocardium tissues, as visualized by histostaining.

FIG. 39 shows the effect of the scaffold on angiogenesis after the scaffold alone, the scaffold loaded with a growth factor, and the scaffold loaded with both a growth factor and cardiac stem cells were transplanted into myocardium tissues, as investigated by an SMA positive vessel formation analysis.

FIGS. 40A-40F show the PLLA fibrous porous 3D scaffold of Example 7-(2), loaded with cardiac stem cells (PLLA), and the PLLA fibrous porous 3D scaffold of Example 7-(2), loaded with cardiac stem cells and VEGF (PLLA/VEGF) in scanning electronic micrographic images after incubation for 1 day (a, d), for 5 days (b, e), and for 7 days (c, f).

FIG. 41 shows the growth of the cardiac stem cells loaded to the PLLA fibrous porous 3D scaffold of Example 7-(2) which contained or did not contain VEGF after incubation for 1, 5 and 7 days.

FIGS. 42A-42B show tissue regeneration effects of the fibrous porous 3D scaffolds of Example 7-(2) which were loaded with neither a growth factor nor stem cells (PLLA), with VEGF (PLLA/VEGF), and with both VEGF and cardiac stem cells (PLLA/VEGF/rCSCs), in terms of fibrotic area (a) and LV wall thickness (b).

FIG. 43 shows tissue regeneration effects of the fibrous porous 3D scaffolds of Example 7-(2) which were loaded with neither a growth factor nor stem cells (PLLA), with VEGF (PLLA/VEGF), and with both VEGF and cardiac stem cells (PLLA/VEGF/rCSCs), in terms of EF (ejection fraction) (a) and FS (fractional shortening) (b).

FIG. 44 shows cell viability and retention rate in tissues of myocardium-defected animal models to which cardiac stem cells were directly injected, or to where a cardiac stem cell-loaded fibrin gel or a cardiac stem cell-loaded fibrous porous 3D scaffold was transplanted.

FIGS. 45A-45D show SEM images of the oriented 3D scaffold of the present invention (a), the scaffold coated with a pDNA complex for transfection (b), and the scaffold 24 hrs after rat cardiac stem cells were loaded thereto (c), and a fluorescence image of the surface of the scaffold (d).

FIG. 46 shows the human VEGF expression in rat cardiac stem cells in confocal images after a plasmid DNA was transfected into the cells.

FIG. 47 is a graph in which cumulative release of DNA from the scaffold of the present invention is plotted against time.

FIG. 48 is a graph showing the expression levels of the hVEGF gene transfected into rat cardiac stem cells in scaffold loaded with the cardiac stem cells alone (Control), with pVEGF (Bolus delivery), and with both cardiac stem cells and pVEGF (Sustained release).

### MODE FOR THE INVENTION

Practical and preferred embodiments of the present invention are illustrated as shown in the following Examples.

However, it will be appreciated that those skilled in the art, on consideration of this disclosure, may make modifications and improvements within the spirit and scope of the present invention.

### Example 1: Preparation of a Polymer PLLA Fiber

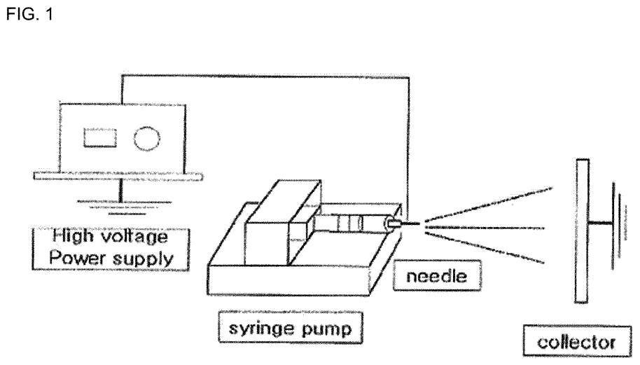

A high molecular PLLA polymer was dissolved in 10 mL of dichloromethane solution, resulting in a 5-10% spinning solution. A fiber was prepared from the spinning solution by electro-spinning (FIG. 1).

As an electro-spinner, DH High Voltage Generator (CPS-40KO3VIT, Chungpa EMT, Korea) was used and the details of the electrospinning process are illustrated with the reference to FIG. 1.

The 5-10% polymer PLLA solution (spinning solution) was filled in a spinning solution depository, which was a 10 mL glass syringe. A needle with blunt tip, which is 0.5-1.2 mm in diameter, was used. The releasing speed of the spinning solution was adjusted to 0.060 ml/min. Voltage was set at 10-20 kV and the electric field distance was adjusted to 10-20 cm. It was important for the entire solvent to be volatilized before the drip of the solution on a collector to prepare a target fiber. Thus, the temperature and humidity had to be carefully regulated; the optimum temperature was 15-20° C. and the optimum humidity was 10-40%.

The prepared polymer PLLA fiber was confirmed to be 3-10 μm in diameter.

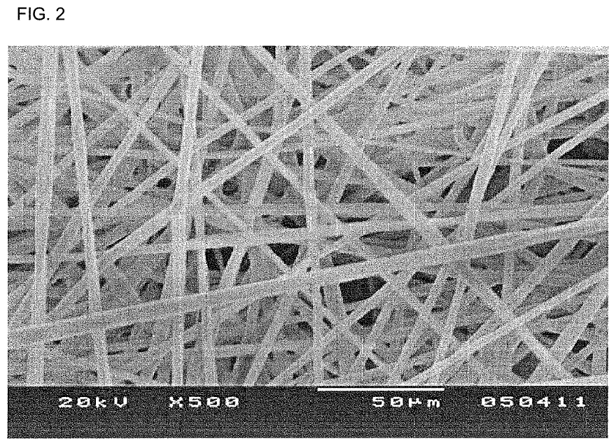

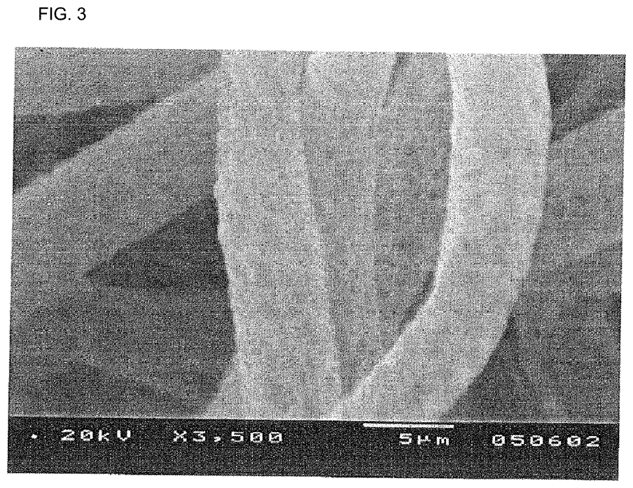

FIGS. 2 and 3 are photomicrographs (×500, ×3500) of fibers prepared under the conditions of 20 cm of double electric field distance, 10 V of voltage, 0.060 ml/min of releasing speed and 1.2 mm of the internal diameter of a needle.

### Example 2: Preparation of a Low Molecular PLLA Fiber

A low molecular PLLA was dissolved in 10 mL of dichloromethane solution, resulting in a 14-20% spinning solution. A fiber was prepared from the spinning solution by electrospinning (FIG. 1).

As an electro-spinner, DH High Voltage Generator (CPS-40KO3VIT, Chungpa EMT, Korea) was used and the details of the electrospinning process are illustrated with the reference to FIG. 1.

The 14-20% low molecular PLLA solution (spinning solution) was filled in a spinning solution depository, which was a 10 mi glass syringe. A needle, which is 0.5-1.2 mm in diameter, was used. The releasing speed of the spinning solution was adjusted to 0.060 ml/min. Voltage was set at 10-20 kV and the electric field distance was adjusted to 10-20 cm. It was important for the entire solvent to be volatilized before the drip of the solution on a collector to prepare a target fiber. Thus, the temperature and humidity had to be carefully regulated; the optimum temperature was 15-25° C. and the optimum humidity was 10-40%.

The prepared low molecular PLLA fiber was confirmed to be 5-10 μm in diameter.

FIG. 2 is a photomicrograph (×2000) of a fiber prepared under the conditions of 10 cm of double electric field distance, 10 V of voltage, 0.060 ml/min of releasing speed and 1.2 mm of the internal diameter of a needle.

### Example 3: Preparation of a Spinning Solution Using Dichloromethane and 1.1.1.3.3.3-Hexafluoroisopropylpropanol

To dichloromethane was added 1,1,1,3,3,3-hexafluoroisopropylpropanol by 2% of the total solvent, resulting in dichloromethane solution. Then, polymer and low molecular PLLA were dissolved in the dichloromethane solution to prepare a spinning solution with proper concentrations of the polymer and low molecular PLLA. A fiber was prepared from the spinning solution by electrospinning. The resultant fiber was proved to be very stable in shape and spun at a wide range of temperature and humidity (possibly spun even at 30° C. with 50% humidity). The obtained polymer was confirmed to be 1-10 μm in diameter. The addition of 1,1,1, 3,3, 3-hexafluoroisopropylpropanol caused the fiber to be thinner and more stable spinning, but at the same time, increased electrostatic force between fibers and formed a shield-like membrane.

### Example 4: Preparation of a Spinning Solution Using Dichloromethane and Acetone

To dichloromethane was added acetone by 10% of the total solvent, resulting in dichloromethane solution. Then, polymer and low molecular PLLA were dissolved in the dichloromethane solution to prepare a spinning solution with proper concentrations of the polymer and low molecular PLLA. A fiber was prepared from the spinning solution by electrospinning. The resultant fiber was proved to be very stable in shape and spun at a wide range of temperature and humidity (possibly spun even at 30° C. with 50% humidity). However, no changes in diameter were observed. The addition of acetone results in the same fiber as obtained by using dichloromethane alone and stabilized the spinning better, suggesting that the added acetone could supplement sensitive factors to enhance the efficiency.

### Example 5: Preparation of a Fluffy 3D Electrospun Scaffolds Using High Molecular Weight PLLA and the Optimization of the Preparation Condition

(1) Preparation of Electrospun Scaffolds

To investigate the effect of the polymer concentration on electrospun fiber morphology, PLLA (intrinsic viscosity 0.63 dL/g, Mw=2.5×10⁵ g/mol) solutions with concentrations ranging from 2 to 10% by weight were prepared in MC/acetone (90:10 v/v). To study the influence of the solvent properties on the interfiber bonding in PLLA meshes, three different solvent compositions were prepared. Eight percent PLLA was dissolved in organic solvent mixtures composed of MC/HFIP, MC/DMF, and MC/acetone, with volume ratios of 90/10. The polymer solution was placed into a 10-mL glass syringe, capped with a 25-gauge blunt end needle, for dispensing at a speed of 0.1 mL/min. The electrospinning process was carried out in a sterile environment at high voltage. A voltage between 8 and 20 kV was used for all solutions. The distance between the needle tip and the collector was 15 cm. Electrospun fibers were collected on a metal plate and formed nonwoven microfibrous mats that were 700 μm in thickness. Before usage, the electrospun scaffolds were dried for 3 days under a vacuum at 70° C. The 3D scaffolds having a depth of 5 mm were prepared by mechanical expansion of as-spun microfibrous mats. As-spun fibrous mats were expanded mechanically into high porosity mats using a metal comb in all directions. After expansion, their volume increased up to about seven times. To prepare nanofibrous membranes as controls, PLLA was dissolved at 3% w/v in a mixture of MC and HFIP (90:10 v/v). Nanofibrous membranes of 300 lm in thickness were prepared using a 16-kV electric field strength and a flow rate of 0.06 mL/min.

(2) Fabrication of 3D Electrospun PLLA Scaffolds

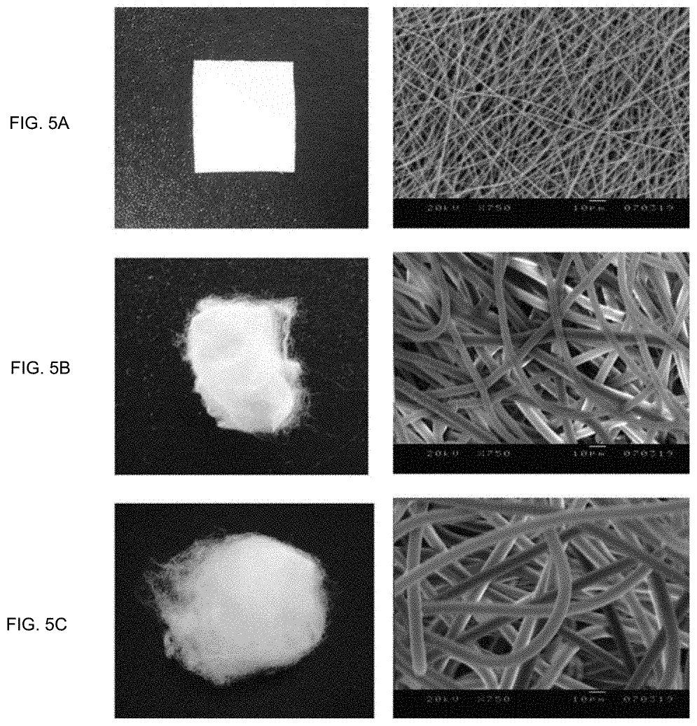

FIG. 5 shows optical and SEM images of electrospun fibrous scaffolds. FIG. 5(a) shows nanofibrous membranes prepared using 3% w/v PLLA solutions in a mixture of MC and HFIP (90:10 v/v), and FIG. 5(b) shows microfibrous mats prepared using 8% w/v PLLA solutions in a mixture of MC and acetone (90:10 v/v). The SEM images of the nanofibrous membranes presented a fibrous morphology, comprising a thin and bonded fiber network of ˜400 nm in diameter (FIG. 5(a)). FIG. 5(b) shows an as-spun fibrous mat with micron-sized fibers of about 7 μm in diameter. The subsequent mechanical expansion of this microfibrous mat led to a 3D fluffy PLLA porous scaffold (FIG. 5(c)). The 3D microfibrous scaffolds of the present invention were prepared after expansion in all directions, resulting in a 7-fold volume increase.

In order that the fibrous mat could be mechanically expanded without breaking the fibers, it is important to fabricate the meshes with little interfiber bonding. Thus, the effects of the solution concentration on the fiber formation were investigated.

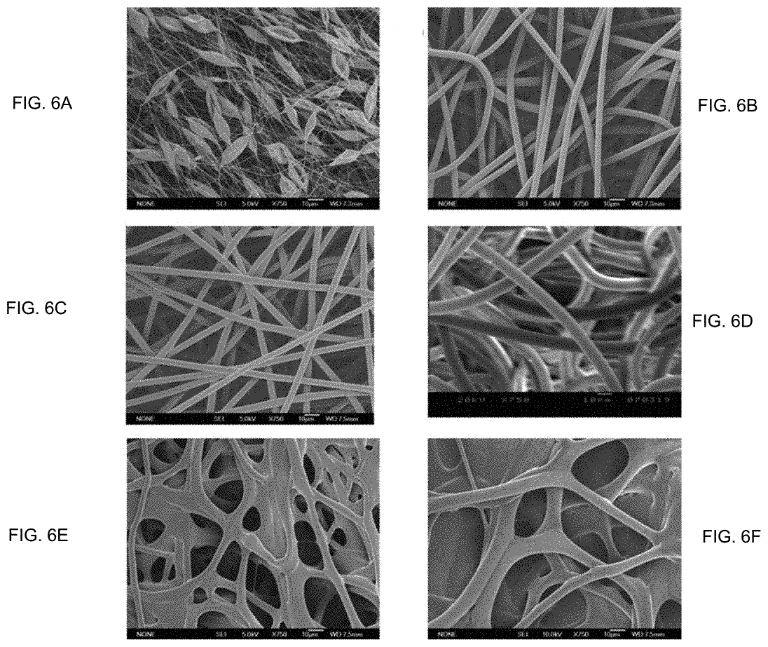

FIG. 6 shows the effects of the solution concentration on the fiber formation using a constant air gap (15 cm), flow rate (0.1 mL/min), and solvent mixture (MC:acetone=90:10 v/v).

As a result, for concentrations below 2% w/v, beaded fibers were observed (FIG. 6 (a)). For concentrations greater than 4% w/v, continuous fibers were spun, the diameters of which increased with increasing polymer concentrations (FIG. 6 (b-d)]. For polymer concentrations greater than 8% w/v, there was failure in the electrospinning process characterized by a fused structure due to large viscosities (FIG. 6 (e,f)). FIG. 6 (c,d) show the rough nanoporous surface of the electrospun microfibers.

Further, the influence of the solvent properties on the electrospinning process was also investigated.

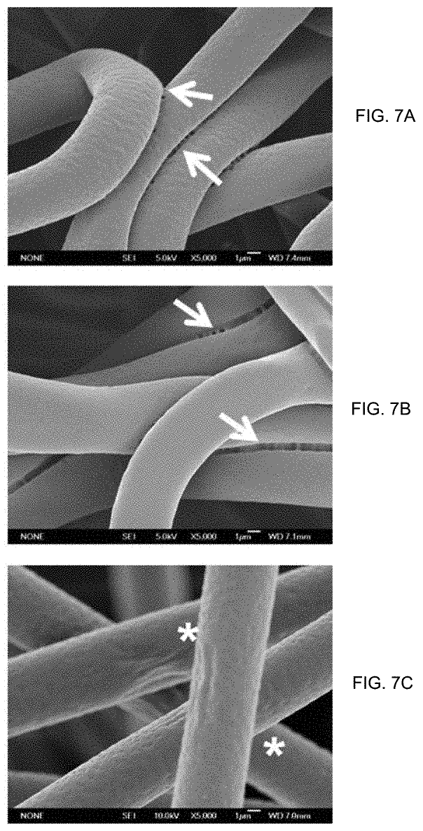

FIG. 7 shows the influence of the solvent properties on the electrospinning process.

As a result, the 8% PLLA was dissolved in organic solvent mixtures composed of MC/HFIP, MC/DMF, and MC/acetone. The PLLA fibers, which were electrospun from the MC/HFIP and MC/DMF mixtures, were fused at the contact point of the fibers (FIG. 7(a,b)); The arrow indicates the fused points.]. The PLLA fibers obtained from the MC/acetone mixture (90:10 v/v) did not show fusion at the contact points (FIG. 7 (c), asterisk). These results suggest that the MC/acetone is the most suitable solvent for preparation of the fluffy 3D porous scaffold of the present invention.

(3) Porosity of the PLLA Scaffolds

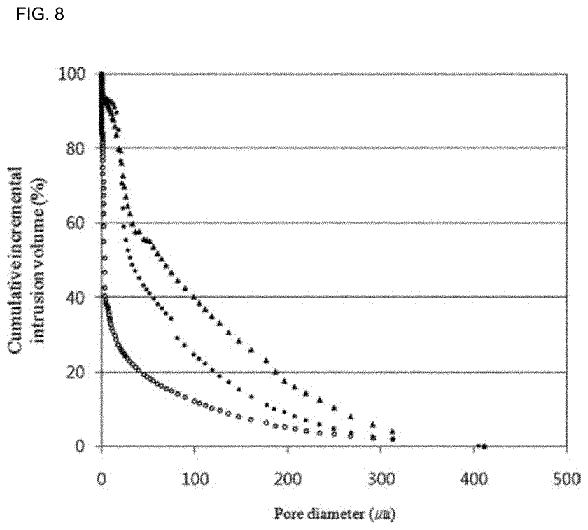

The volumetric porosity and pore size, namely the distance between the fibers in the fibrous scaffold, were measured using mercury porosimetry. FIG. 8 shows the relationship between the cumulative incremental intrusion volume and the pore diameter for the electrospun fibrous scaffolds.

Nanofibrous membranes, as-spun microfibrous mats, and fluffy 3D microfibrous scaffolds of the present invention showed mean pore sizes of 68.6, 147, and 206 μm and porosities of 85.9, 91.3, and 96.4%, respectively.

### Example 6: Examination of Suitability of a Fluffy 3D Electrospun Scaffolds on Bone Regeneration

(1) Osteoblast Adhesion Test

The following experiment was performed to investigate the adhesion capacity of the fluffy 3D porous electrospun scaffold of the present invention.

The scaffolds prepared by expanding fibrous mats prepared in Examples 1 and 2 mechanically were sterilized with 70% ethanol, on which sub-cultured osteoblasts (MC3TC-E1) were static cultured. Observation on the adhered cells was performed under differential scanning microscope.

The cells remaining without being adhered were eliminated. 25% (w/w) glutaraldehyde was diluted in 0.1 M phosphate buffered saline (PBS, pH 7.4), resulting in 2.5% glutaraldehyde solution, with which pre-fixation was carried out for 4-20 minutes. After the fixation, water was eliminated by using ethanol, followed by freeze-drying. Then, the sample was coated with gold and observed under differential scanning microscope.

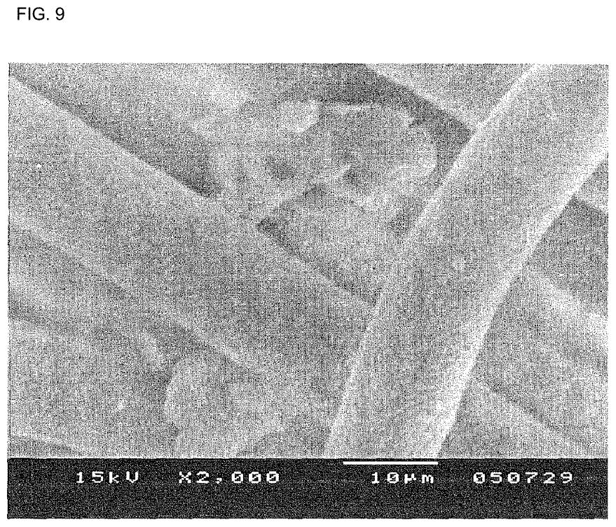

As a result, the prepared fiber was still stable in shape and in strength even after 7 days from the preparation and osteoblasts were packed between and on the surfaces of the fibers. Accordingly, it was confirmed that the scaffold of the present invention had cellular affinity, so that cells could be adhered stably. Therefore, the porous scaffold of the invention can be accepted as an appropriate scaffold material (FIGS. 8 and 9).

(2) Suitability of a Fluffy 3D Porous Electrospun Scaffolds Prepared in Example 5-(1) on Bone Regeneration

1) Experimental Procedure

Cell Culture

MC3T3-E1 cells were cultured in a-MEM medium, supplemented with 10% FBS and 1% antibiotic solution, in a humidified atmosphere containing 5% CO2 at 37° C. After reaching confluence, the cells were washed with phosphate-buffered saline (PBS) and detached using Trypsin-EDTA (0.25% Trypsin in 0.04 mM EDTA). Viable cells were counted using a trypan blue assay and were suspended in complete medium. All of the sample scaffolds were sterilized in a 70% ethanol solution for 10 min prior to the cell culture, and then placed in 24-well plates. The samples were then exchanged two times (30 min each) with PBS and extensively washed with a-MEM. The cells were seeded onto the scaffolds inside 24-well plates at a density of 2×10⁵ cells/matrix and incubated at 37° C. for 1.5 h, in a humidified atmosphere of 5% CO2. Complete culture medium including 10 mM sodium b-glycerol phosphate, 50 μg/mL L-ascorbic acid, and 10⁻⁷M dexamethasone (1 mL) was then added to each well and the culture was continued for 24 h. The culture was maintained at 37 C and 5% CO2, and the medium was replaced every day.

Cell Morphology

The MC3T3-E1 cells were seeded on nanofibrous membranes and microfibrous scaffolds, respectively. After 1, 7, and 14 days of culture, the morphologies of the cells were observed using SEM. The cells adhering to the samples were washed with PBS and then fixed in PBS for 20 min at 4° C. using 2.5% glutaraldehyde. After thoroughly washing them twice with PBS (10 min each time), the cells were postfixed using 1% OsO₄ in PBS for 20 min at 4° C. In addition, the fixed samples were sequentially dehydrated for 10 min using 70, 80, 90, and 95% ethanol solutions and finally 100% ethanol (two treatments of 10 min each). The samples were subsequently treated twice with hexamethyldisilazane (30 min each) and kept in a fume hood for air drying.

Histochemical Assessment

Scaffolds were first fixed with 4% paraformaldehyde buffered saline (pH 7.4). The specimens were decalcified with a 10% EDTA solution for 2 weeks, dehydrated using a series of ethanol solutions with increasing concentrations, and embedded in paraffin. Five-micron-thick coronal sections through the center of the circular defects were obtained and stained using hematoxylin and eosin and then washing with tap water. These specimens were finally examined using light microscopy.

Cell Proliferation Assay

After 1, 3, 7 and 14 days of culture, cell proliferation assays were performed with the Cell Counting Kit-8 (CCK-8) (Dojindo, Kumanoto, Japan). Cell numbers in triplicate cells caffold constructs were measured as the absorbance (450 nm) of reduced WST-8 (2-(2-methoxy-nitrophenyl)-3-(4-nitrophenyl)-5-(2,4-disulfophenyl)-2H-tetrasolium, monosodium salt). For this assay, the culture medium was removed, and cell-scaffold constructs were washed with PBS before the addition of serum-free medium plus CCK-8 solution. The samples were then incubated for 3 h at 37° C. in a humidified atmosphere of 5% CO2. The incubated medium (200 μL) was transferred to a 96-well culture plate, and the optical densities were measured using a microplate reader. To calculate the cell numbers, standard curve were plotted using serial dilutions of same MC3T3-E1 cells.

Alkaline Phosphatase (ALPase) Activity Assay

As an early marker of osteoblastic differentiation, the ALPase activity of the cells cultured on scaffolds was examined by measuring the conversion activity of p-nitrophenyl phosphate into p-nitrophenol after 3, 7, and 14 days of cultivation.

The process material for the analysis of the ALPase activity, 250 μL of a cell lysis solution of protease inhibitors (2.0 μg/mL aprotinin, 2.0 μg/mL leupeptin, 1.0 μg/mL pepstatin, Calbiochem, La Jolla, Calif.) was added to each cellscaffold construct. Each supernatant (50 μL) was then incubated with ALPase reagent (Sigma, Cat #245) at 37° C. for 30 min. An estimation of the protein content was performed using the BCA assay method (BCA protein assay reagent kit, Pierce Chemical Co., Rockford, Ill.). The ALP activity was determined based on the release of p-nitrophynol from pnitrophenylphosphate, so that the specific activity could be calculated. The production of p-nitrophenol in the presence of ALP activity was measured using a spectrophotometric plate reader at 405 nm. The ALPase activity was expressed in terms of units per gram of protein.

In Vivo Bone Formation Study

New Zealand White male rabbits weighing between 2.5 and 3 kg (n=4 per test group) were used to assess the in vivo bone forming capacity of electrospun microfibrous matrices. The rabbits were anesthetized by an intramuscular injection of ketamine hydrochloride (10 mg/kg). After wiping the surgical site with betadine, local anesthesia was provided with a 2% lidocaine solution. The skin and subcutaneous tissues were separated from the periosteum using blunt dissection. A second longitudinal incision was made through the periosteum, which was elevated and carefully dissected from the underlying skull bone. A craniotomy defect (8 mm in diameter) was then formed using a trephine bur in a dental hand piece, while being supplemented with physiological saline. After dissecting the calvarial disc, the samples were placed into the defect, and the soft tissues and skin were closed using a 5-0 chromic gut and 4-0 silk (Ethicon, Somervile, N.J.). Disc-shaped fibrous matrices of 8 mm in diameter were used with rabbit calvarial defects for animal testing. The animals were sacrificed 2 and 4 weeks after the implantation. The retrieved specimens were fixed in a formalin solution, decalcified in a 5% trichloroacetic acid solution, and embedded in paraffin. Coronal sections (5 μm in thickness) were sliced and stained using hematoxylin-eosin (Sigma). Microscopic examination was conducted using an Olympus BH-2 optical microscope (Olympus Optical Co., Osaka, Japan). The NIH guidelines for the care and use of laboratory animals were followed for all animal experiments.

2) Results

In Vitro Cell Culture

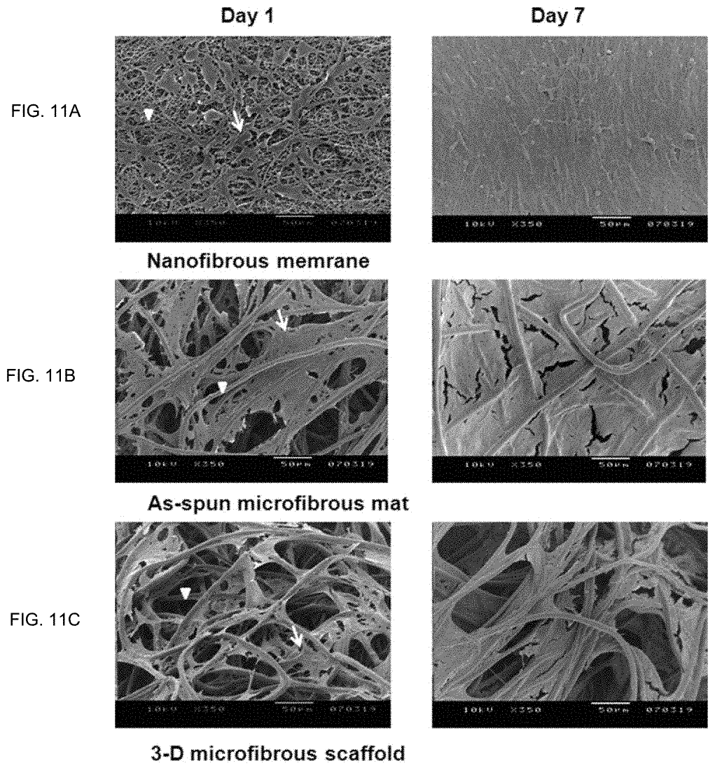

To investigate the effects of electrospun fiber geometry on osteoblast proliferation and differentiation, nanofibrous membranes, microfibrous mats, and mechanically expanded fibrous scaffolds of the present invention were prepared. Nanofibrous membranes were fabricated using 3% PLLA solutions and a mixture of MC and HFIP (90:10 v/v), using electrospinning with an electric field strength of 16 kV and a flow rate of 0.06 mL/min. Microfibrous mats were prepared using 8% w/v PLLA solutions and a mixture of MC and acetone (90:10 v/v), and 3D microfibrous scaffolds were fabricated after a subsequent mechanical expansion. After the culture on fibrous scaffolds for 1 and 7 days, morphological changes of the MC3T3-E1 cells were observed, namely the transition from their original round shape to an elongated spindle-like shape. On the nanofibrous membranes, the cells were attached to the surface and spread over the fibers at day 1, and they were distributed on the surface only as a quasimonolayer at day 7 (FIG. 11 (a)). In the microfibrous mats, the cells were attached to the surface, but their penetration was limited to inside the matrices (FIG. 11(b)). At day 7, the cells had proliferated along the surface fiber network of the microfibrous mat. In contrast, the cells were attached to both the surface and the inside of the matrices when they were cultured on the 3D microfibrous scaffolds (FIG. 11(c)).

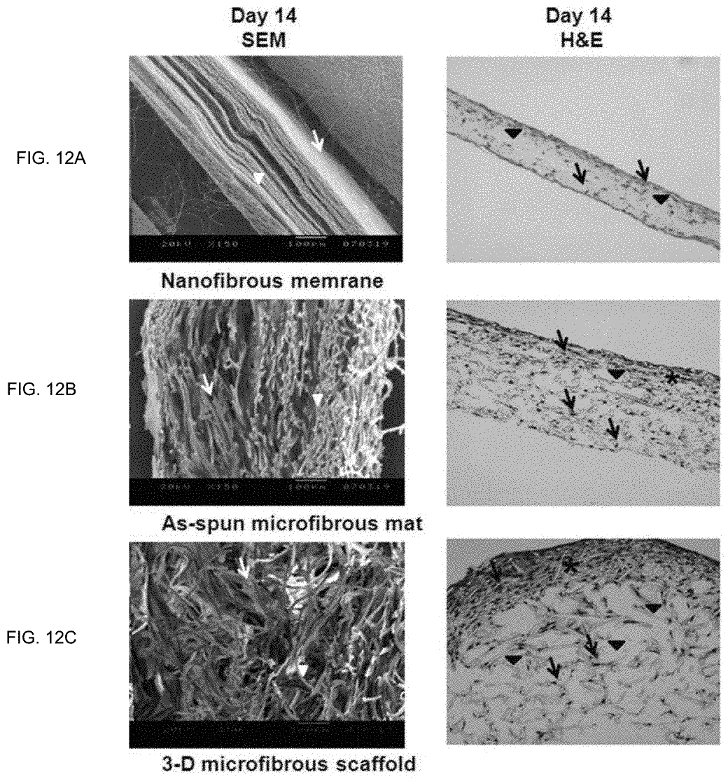

An overview of the whole cross-section of the cell-scaffold constructs is shown in FIG. 12 after 14 days. Cells penetrate throughout both the microfibrous scaffolds (FIG. 12(b,c)). In addition, the use of the mechanical expansion in the fabrication process led to thick 3D fibrous scaffolds and a complete cell infiltration with sufficient scaffold thickness (FIG. 12(c)). In contrast, the cells could not penetrate inside the nanofibrous matrix (FIG. 12(a)), and a few of them were found inside the microfibrous mat (FIG. 12(b)].

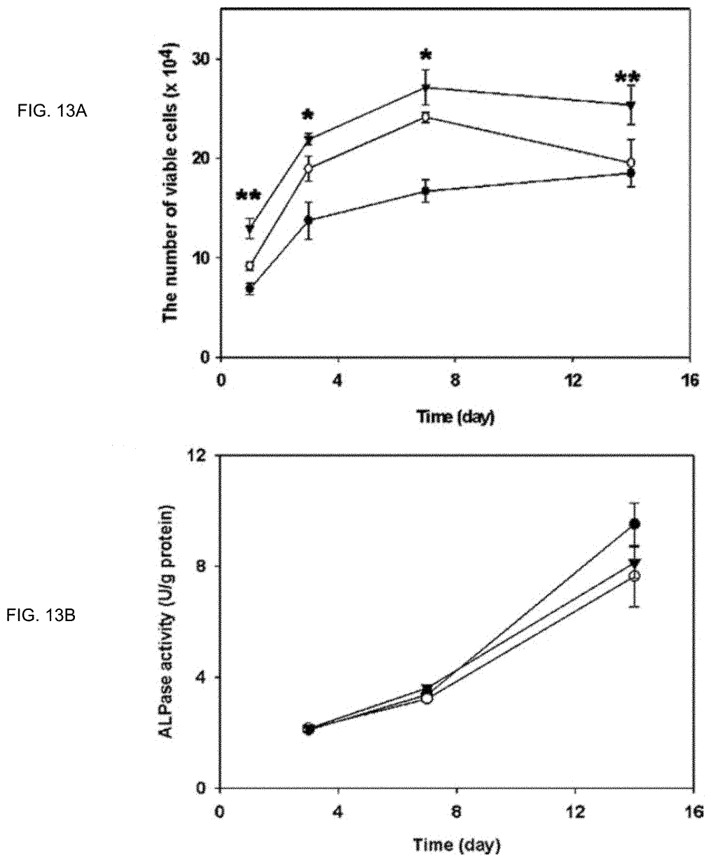

The cell numbers were measured using a CCK-8 assay (FIG. 13(a)). The level of cell attachment to the 3D microfibrous scaffolds was statistically higher than that to the microfibrous mats and nanofibrous membranes (p<0.05), with 65% attachment compared to the initially applied cell density at day 1. The cell proliferation and ALPase tests were repeated three times. The slope of the proliferation curve for the mechanically expanded 3D microfibrous scaffolds was the highest among all fibrous scaffolds. Viable cell numbers were statistically higher compared to the cells cultured on nanofibrous membranes at day 3 and 7 days and cultured on both nanofibrous membranes and as-spun microfibrous mats at day 1 and 14 days. The culture of osteoblasts on all of the substrates revealed that the activity of the ALPase enzymes was sufficient for detection at days 3, 7, and 14 (FIG. 13(b)). The amount of ALPase was similar on all scaffolds for each time interval.

In Vivo Bone Formation

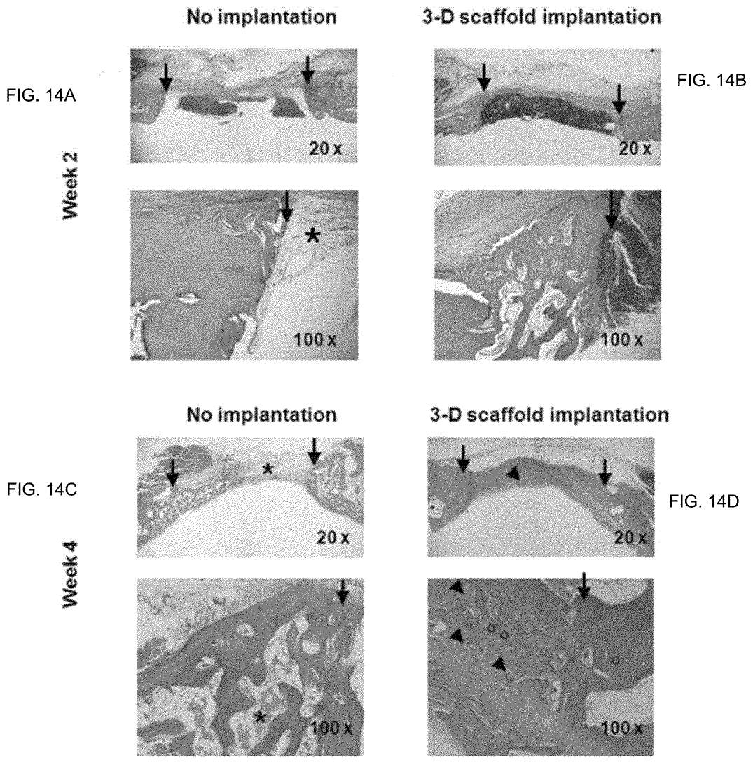

FIG. 14 shows the histological sections of the rabbit calvarial defects corresponding to the electrospun 3D microfibrous matrices after an implantation of 2 and 4 weeks. For the negative control group, where no matrix implantation was applied, the defect sites were filled by loose, fibrous, connective tissues after 2 weeks (FIG. 14(a)). Although new bone formation along the defect margin was observed without scaffold at 4 weeks, it might not be directly related to the implant (FIG. 14(b)). Because of the interconnected porous structure, we observed that the cells and tissues penetrated throughout the electrospun 3D microfibrous scaffolds after 2 weeks (FIG. 14(b)). But most of cells were connective tissues and inflammatory cells were observed at 2 weeks. The presence of osteoblasts inside the scaffold and the formation of new bone (arrowhead) were observed after 4 weeks. The residual scaffold material was visible for the treated defects of the scaffold after 4 weeks (circle symbols).

### Example 7: Effect of Fluffy 3D Porous Electrospun Scaffold Loaded with Stem Cells or Gene

(1) Preparation of Stem Cell-Loaded Fluffy 3D Porous Electrospun Scaffold 1

1) Preparation of Fluffy, Fibrous, Porous 3D Scaffold in which Separable Fibers are Entangled.

Poly-L-lactic acid (PLLA) (Inherent viscosity: 1.8 dL/g, Purac, Inc.,) was dissolved in dichloromethane/acetone (acetone used in 10-40% by volume of total volume of the solution; exact 20% by volume in this example) to form a solution containing a solid content of 14-20% (exactly, 15% in this example).

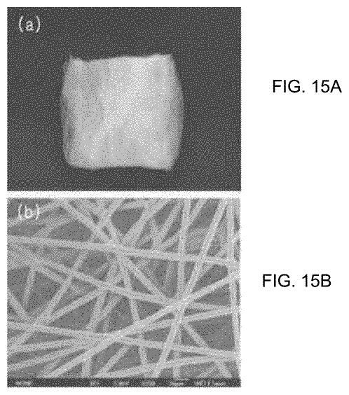

The solution was electrospun using the electrospinner DH High Voltage Generator (CPS-40KO3VIT, Chungpa EMT, Korea) at a spinning speed of 0.06 ml/min, a voltage of 10 kV, and an electric field distance of 15 cm. Electrospining was conducted at a temperature of 15-25° C., and a humidity of 10-40% such that the solvent was volatilized, prior to the deposition of the fibers on the stainless steel plate collector, to prepare a microfibrous mat in which the biodegradable fibers were separable and entangled in a network structure. The microfibrous mat was about 300 μm thick with a fiber thickness of about 7 μm. Thereafter, one or more physical forces were applied in opposite directions to the microfibrous mat to increase the total volume and expand pores between the entangled fibers to afford a fluffy scaffold. This fluffy 3D porous scaffold obtained from the microfibrous mat by application of a physical force was 1 mm or more thick and had a pore size of 50-300 μm with a porosity of 50-90% (FIG. 15).

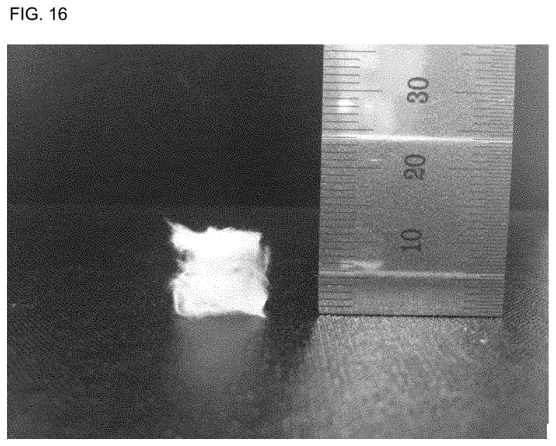

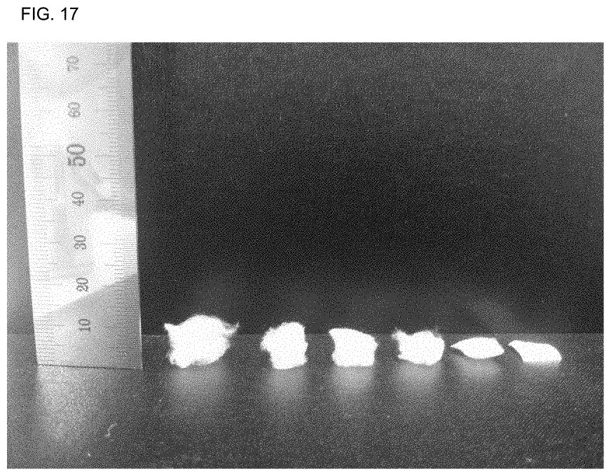

To examine the flexibility of the fluffy scaffold, its thickness was measured after one or more forces were applied in opposite directions to the electrospun scaffold to increase the total volume and expand pores between fibers. As a result, the 3D scaffold for tissue regeneration of the present invention was observed to expand to 1 cm or thicker (FIG. 16), demonstrating its complete difference in syntax and flexibility from a 2D membraneous microfibrous mat (FIG. 17).

Having the structure in which fibers are separable from each other and in a fluffy scaffold, the scaffold of the present invention can alternatively expand or shrink in response to the proliferation and migration of cells, or to the swelling or contraction of the tissues to which the fibers are attached.

2) Seeding of Cardiac Stem Cells

To seed cardiac stem cells thereinto, the fibrous porous 3D scaffold were sterilized with 70% ethanol, and washed with a buffer. After dehydration, a medium in which 1×10⁶-1×10⁸ cells (5×10⁶ cells used in this Example) were suspended was loaded to the scaffold and allowed to stand for 1 hr or longer. Subsequently, the the scaffold was incubated at 37° C. for 24 hrs in a 5% CO2 atmosphere to stabilize the cell adhesion. The cells were cultured in DMEM (Dulbecco's Modified Eagle's Medium) supplemented with 1% penicillin, 10% fetal bovine serum, and 10 ng/ml human EGF at 37° C. under 5% CO2 with saturated humidity.

(2) Preparation of Stem Cell-Loaded Fluffy 3D Porous Electrospun Scaffold 2

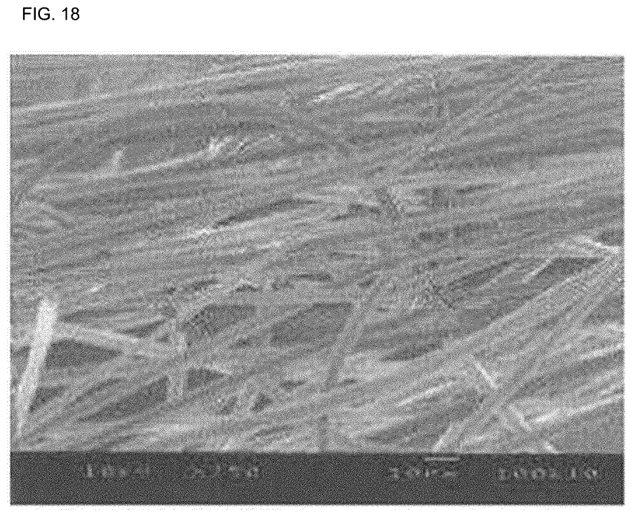

A stem cell-loaded porous 3D scaffold in an oriented network structure was prepared in the same manner as in Example 7-(1), with the exception that a cylindrical drum collector, instead of the stainless steel plate collector, was used at a rotation speed of 2,000 rpm (FIG. 18).

(3) Preparation of Stem Cell- and/or Growth Factor-Loaded Fibrous 3D Scaffold

Vascular endothelial growth factor (VEGF) was seeded into the 3D scaffolds prepared in Examples 7-(1) and 7-(2) to afford fibrous porous 3D scaffolds loaded with stem cells and VEGF.

Vascular endothelial growth factor (VEGF) was seeded into the stem cell-free 3D scaffold of Examples 7-(1) and 7-(2) to afford fibrous porous 3D scaffolds loaded with VEGF.

(4) Preparation of Gene-Loaded Fibrous Porous 3D Scaffold

In a suspension of PLGA in a mixture of acetone/ethanol, aqueous pVEGF/CPP complex or FITC was emulsified, and the emulsion was electrospun onto the surface of the scaffold of Example 7-(2), and lyophilized. Herein, the pVEGF complex was used in a fixed amount of 3 μg per scaffold (8*8).

(5) Test for Adhesion of Cardiac Stem Cells

The following experiment was performed to examine the adhesion of cells to the fibrous porous 3D scaffolds of Examples 7-(1) and 7-(2) after stabilization for 48 hrs.

Unattached cells were washed out before the scaffolds were fixed for 20 min with a 2.5% glutaraldehyde solution. Subsequently, the samples were dehydrated sequentially with 70%, 80%, 90%, and 100% ethanol for 10 min each, and then completely dried in a vacuum. Thereafter, the samples were coated with gold.

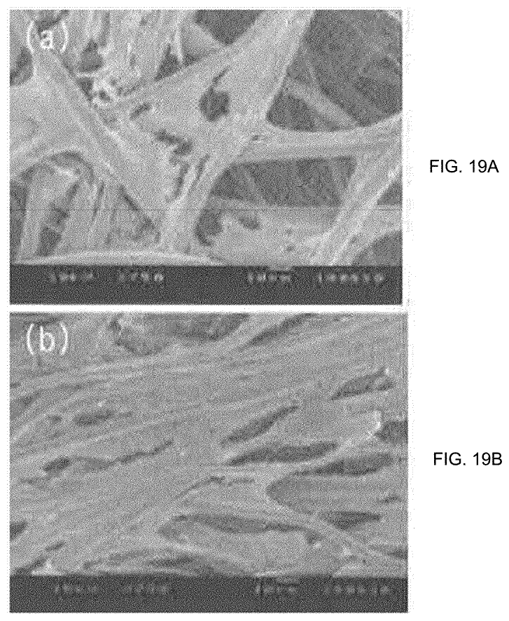

Differential scanning microscopic views of the samples are given in FIG. 19. As can be seen, cells were well attached to the scaffolds. Particularly, in the scaffold in an oriented network structure of Example 7-(2), the cells were arranged in an oriented direction along the fibers (FIG. 19b)

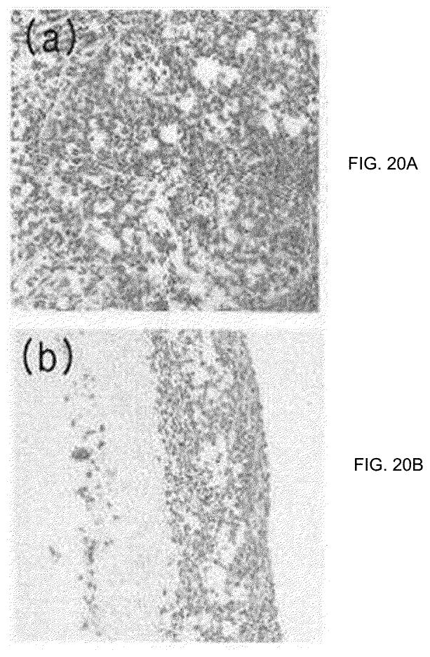

To examine the density of the cells attached, the 3D scaffold-cell aggregate obtained after stabilization for 48 hrs in Example 7-(2) was washed with a buffer and fixed overnight in a 3.7% formaldehyde solution. It was embedded in paraffin, sectioned at 4 μm thickness, and stained with H & E (hematoxylin and eosin) before microscopic observation (FIG. 20).

In a widthwise cross section view, the cells were observed to be attached at a high density (FIG. 20a) while a cross sectional view shows the existence of the cells inside as well as outside the fibers at a high density (FIG. 20b). This is attributed to the fact that the physical expansion increases pores in size to allow for the penetration of the stem cells into the 3D scaffold for tissue regeneration of the present invention.

(6) Distribution and Release of Growth Factor

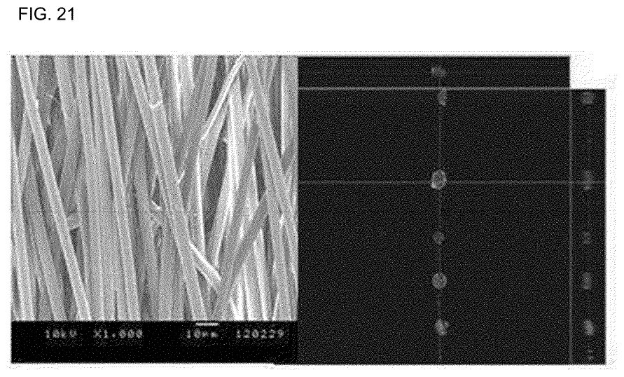

The distribution of the growth factor in the fibrous porous 3D scaffold in an oriented network structure, prepared in Example 7-(3), was examined with a fluorescent reagent. As can be seen in FIG. 21, VEGF was uniformly distributed over the fibers.

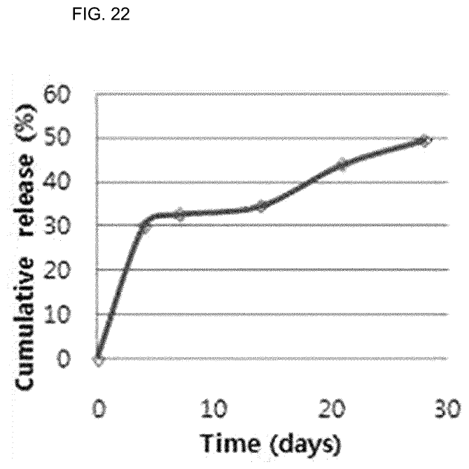

In addition, the growth factor-loaded scaffold was examined for the release and release speed of the growth factor. Cumulative release of the VEGF from the scaffold was plotted over time, indicating that the scaffold can release VEGF in a sustained pattern to angiogenesis to prolong the angiogenesis effect (FIG. 22). That is to say, the growth factor-loaded scaffold of the present invention is able to release locally to a cardiac infarction zone for a prolonged period of time. In addition, when loaded with stem cells and a growth factor, the scaffold of the present invention can release the growth factor in a sustained manner to help the stem cells differentiate into appropriate cells, thereby effectively performing tissue regeneration.

(7) Observation of Cell Proliferation Ex Vivo

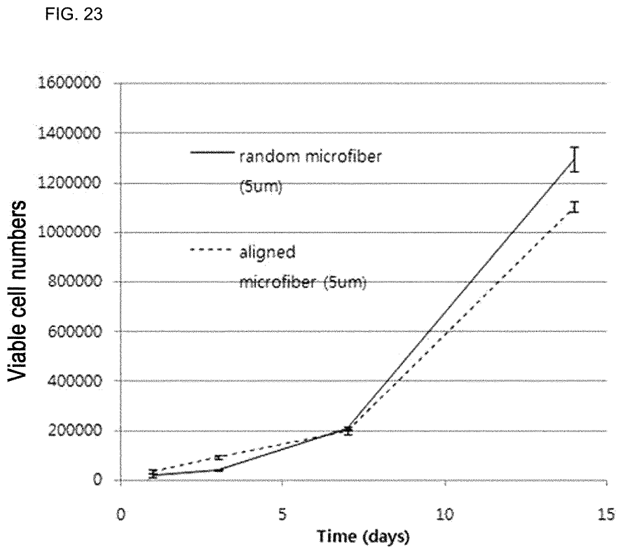

While the stem cell-loaded fibrous porous 3D scaffolds prepared in Examples 7-(1) and 7-(2) were incubated for two weeks in a growth medium, cells were counted on predetermined days, such as day 1, day 4, day 7 and day 14, using Cell Counting Kit-8 (CCK-8) (FIG. 23). As can be seen in FIG. 23, both the scaffolds, which were spun in a random, non-oriented manner (Example 7-(1)) and in an oriented manner (Example 7-(2)), were observed to allow for high cell proliferation.

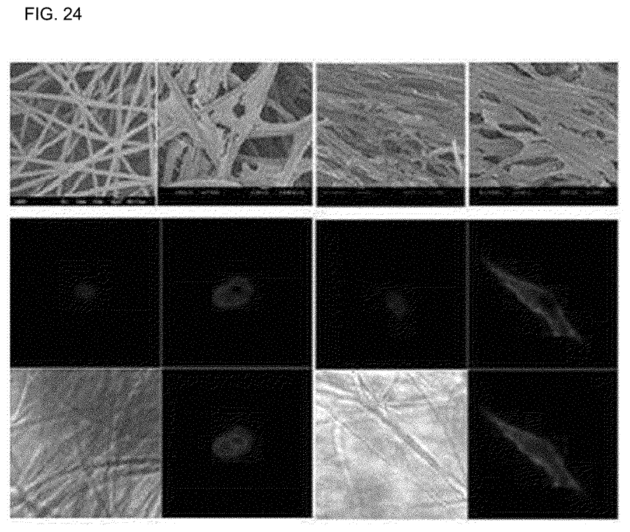

A comparison was made of proliferation pattern between the porous 3D scaffolds of Examples 7-(1) and 7-(2). In contrast to the scaffold of Example 7-(1), the oriented, fibrous porous 3D scaffold for tissue regeneration of Example 7-(2) was found to help the cardiac stem cells proliferate in an oriented direction and in morphology similar to that of the myocardiocytes themselves (FIG. 24).

(8) Observation of Cell Differentiation Ex Vivo

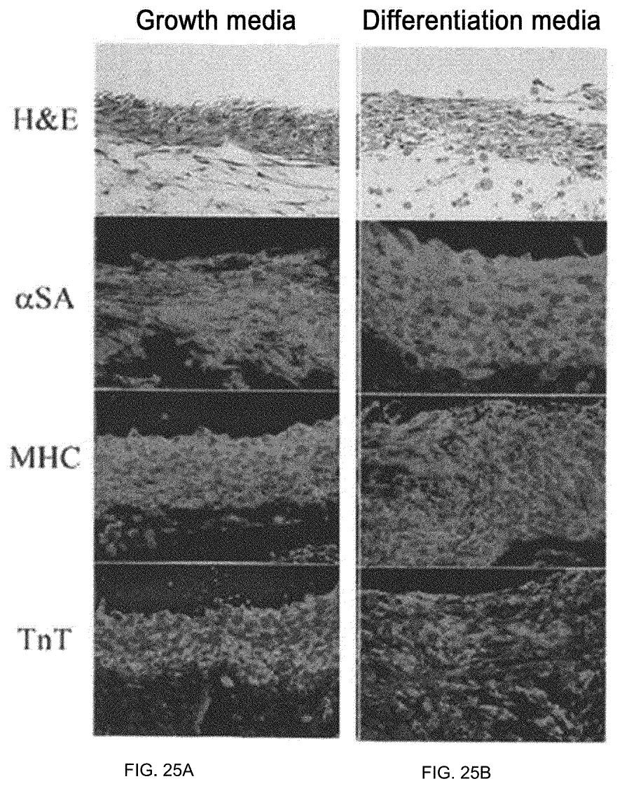

The cardiac stem cell-loaded scaffold of Example 7-(1) was incubated ex vivo for two weeks in a growth medium and in a differentiation medium. The scaffold was fixed in paraffin, sectioned in a lengthwise direction, and stained with H&E before the observation of differentiation into heart cells by microscopy (FIG. 25).

An oriented direction in which the cells were grown was observed. Also, the sarcomeric proteins, α-sarcomeric actinin (αSA), myosin heavy chain (MHC), and troponin-T (TnT), which are involved in the contraction of muscle contraction, were expressed in the corresponding cells.

In the differentiation medium, the cells on the scaffold were found to differentiate into mycocardiocytes at a high rate. Differentiation into myocardiocytes was also observed in the scaffold in the growth medium. This was believed to be attributed to the use of cardiac stem cells.

(9) Observation of Tissue Fusion and Inflammatory Reaction Upon Transplantation

Sprague-Dawley rats (8-9 weeks old) were anesthetized with isoflurane, and ventilated under positive pressure.

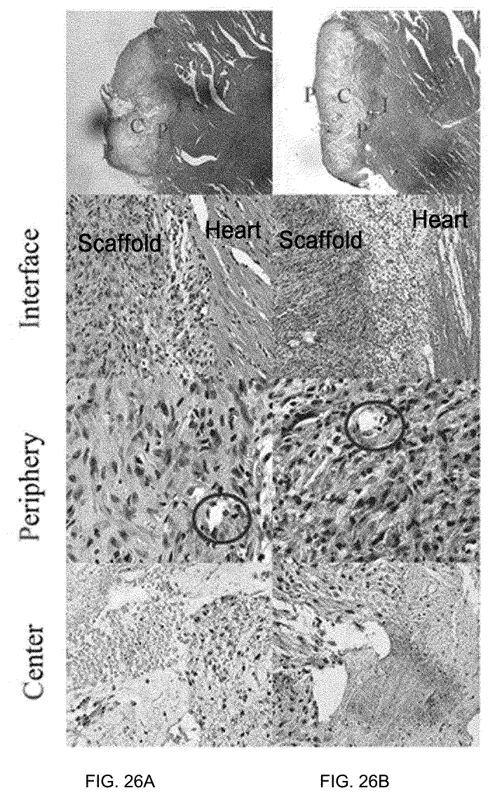

To assess the stability thereof, the stem cell-loaded porous 3D scaffold prepared in Example 7-(1) was transplanted to the surface of the epicardium of a normal heart tissue and incubated for 4 days. H&E staining exhibited fusion to the tissue and the degree of inflammatory reaction, as shown in FIG. 26.

In the panel P (Periphery) of FIG. 26, circles represent blood vessels, demonstrating the growth of blood vessels into the scaffold. That is, the scaffold was histologically fused well to the heart. With regard to cell differentiation, tissue formation was observed at the interface between the normal heart tissue and the scaffold (panel I of FIG. 26), and at the periphery (panel P of FIG. 26), indicating fusion to myocariocytes, as well.

(10) Analysis for Improvement in Heart Function by Transplantation of Stem Cell-Loaded Porous 3D Scaffold into Disease Model 1

1) Establishment of Disease Model (Myocardial Infarction, MI Model)

Sprague-Dawley rats (8-9 weeks old) were anesthetized with isoflurane and ventilated under positive pressure. They were opened between ribs 2 and 3 on the left. After the cardium was cut to push out the heart by thorax compression, the left coronary artery was ligated with a 7-0 Prolene suture to induce cardiac infarction. The ligation of the left anterior descending coronary artery (LAD) was performed, followed by identifying the generation of ischemic regions. Immediately, the stem cell-loaded, or stem cell- and growth factor-loaded scaffold of one of Examples 7-(1) to 7-(3) with a size of 10×10 mm was attached to the infarcted myocardium zone or the infarction border zone, an epicardial surface where infarction was induced by ligation with 7-0 silk suture.

2) Establishment of Disease Model (Myocardial Infarction, MI Model)

MI models were established in the same manner as in Example 7-(10)-1), with the exception that the scaffolds of Examples 7-(1) to 7-(3) were transplanted to the anterior wall of the left ventricle. They were sacrificed on day 28 after the transplantation.

3) Analysis for Improvement in Cardiac Function

(A) Immediately after injury, the stem cell-loaded scaffold of Example 7-(1) and the stem cell-free scaffold of Example 7-(1) (Comparative Example 1) were transplanted to the anterior wall of the left ventricle. On day 14 after transplantation, the models were sacrificed, and the cardiac tissues were fixed in 10% buffered formaldehyde and paraffin sectioned for observation.

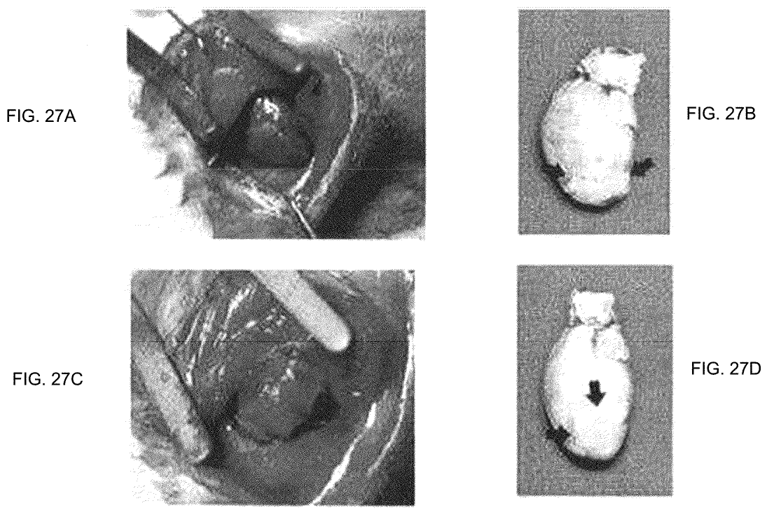

FIG. 27 shows an infarcted myocardium zone immediately after the cardiac stem cell-loaded scaffold of Example 7-(1) was transplanted (a), the cardiac tissue 14 days after the cardiac stem cell-loaded scaffold of Example 7-(1) was transplanted (b), an infarcted myocardium zone immediately after the stem cell-free scaffold of Comparative Example 1 was transplanted (c), and the cardiac tissue 14 days after the stem cell-free scaffold of Comparative Example 1 was transplanted (d).

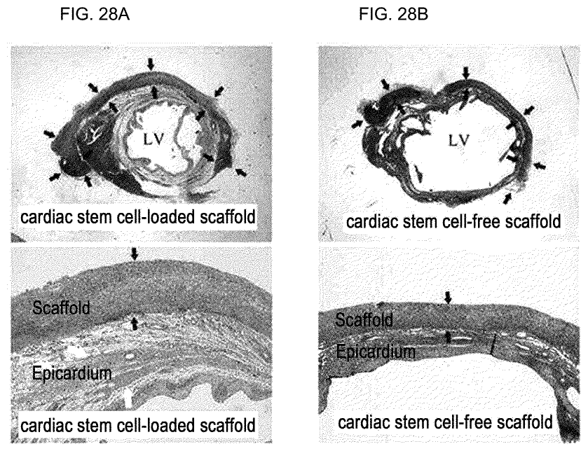

The tissues were paraffin sectioned and stained with H&E (hematoxylin and eosin) (FIG. 28). At 14 days of coronary artery ligation, cardiac infarction was successfully induced, and was also observed to cause myocardium loss and cardiomegaly.

As shown in FIG. 28, the scaffolds of Examples 7-(1) and Comparative Example 1 completely fused to the LV anterior wall. Further, the cardiac stem cell-loaded scaffold of Example 7-(1) (FIG. 28a) retained its morphology well, and made the LV anterior wall thicker than did the cell-free scaffold of Comparative Example 1. After being transplanted with the scaffold of Example 7-(1), the heart was less prone to LV dilatation, compared to the scaffold of Comparative Example 1, and allowed for the remarkable regeneration of cardiac muscles (as indicated by white arrows).

(B) The tissues in which the cardiac stem cell-loaded scaffold was implanted for 14 days according to (A) were stained with a myocardium-specific antibody and then with a fluorescein isothiocyanate-conjugated secondary antibody. They were observed to express marker specific for α-sarcomeric actinin (αSA) and troponin-T (TnT), which are sarcomeric proteins involved in cardiac muscle contraction.

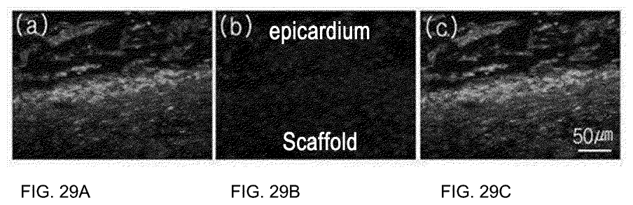

FIG. 29 shows fluorescence images of a cardiac tissue immunostained with αSA (a) and with DAPI (b) for nuclei after transplantation with the scaffold for 14 days, and a merged image (c) thereof, demonstrating that the stem cells within the scaffold differentiated into αSA myocardiocytes.

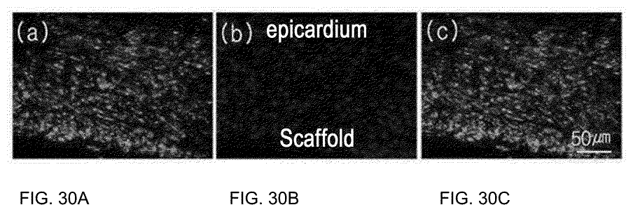

FIG. 30 shows fluorescence images of a cardiac tissue immunostained with TnT (a) and with DAPI for nuclei (b) after transplantation with the scaffold for 14 days, and a merged image (c) thereof, demonstrating the stem cells within the scaffold differentiated into TnT myocardioxytes.

(C) An immunostaining test with smooth muscle alpha actin (SMA) and CD34 implied that angiogenesis was increased in an ischemic site where the scaffold of Example 7-(1) was transplanted for 14 days.

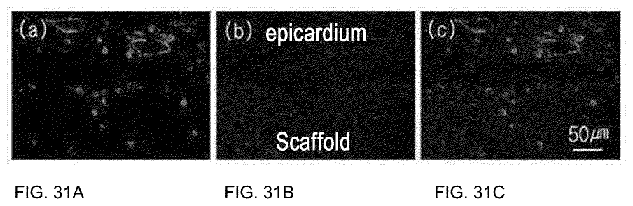

FIG. 31 shows fluorescence images of a tissue immunostained with SMA (a) and with DAPI for nuclei (b) after transplantation with the scaffold for 14 days, and a merged image (c) thereof, demonstrating angiogenesis (generation of arterioles/veins) in the graft scaffold.

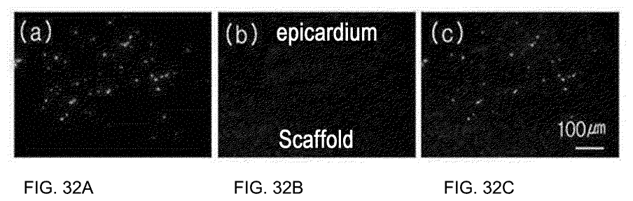

FIG. 32 shows fluorescence images of a tissue immunostained with anti-CD34 antibody (a) and with DAPI for nuclei after transplantation with the scaffold for 14 days, and a merged image (c) thereof, demonstrating the growth of capillary vessels in the graft scaffold.

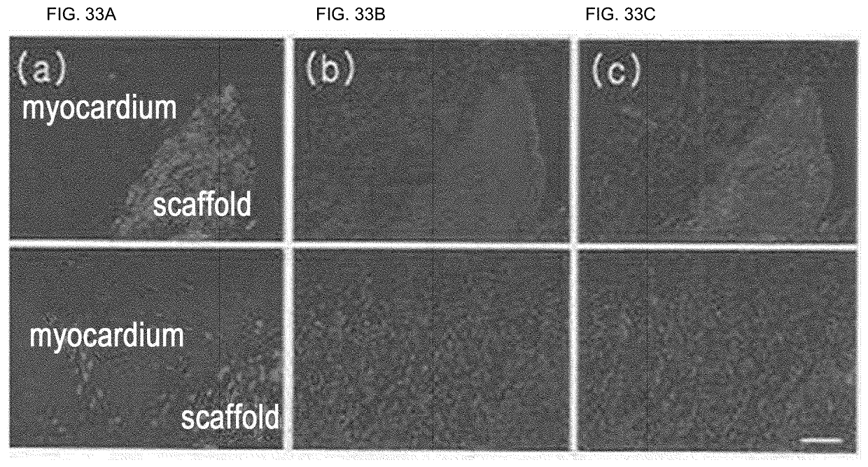

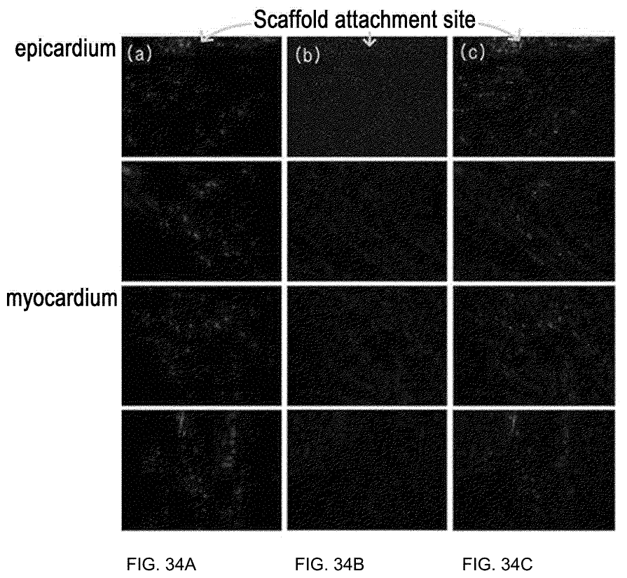

(D) To chase the survival and progression of cells after transplantation, the cells were harvested and labeled with a red fluorescent DiI. Approximately 10⁶ cells were loaded to the scaffold which was then transplanted into an infarcted zone of the heart. The viability of the transplanted stem cells was monitored by fluorescence microscopy for DiI after sacrifice. FIGS. 33 and 34 are a series of microphotographic images showing the locations of the transplanted cardiac stem cells in the myocardium layer of the myocardium infarcted rats 14 days after transplantation.

FIG. 33 shows low-magnification microscopic images of entire scaffold and cardiac tissues transplanted with DiI-labeled cardiac stem cells in red fluorescence (a) and nucleus-stained with DAPI 14 days after transplantation, and a merged image thereof (c) indicative of observation of DiI-positive cell aggregates within the scaffold. Taken together, the data obtained from the images demonstrate that the stem cells survived for a long period of time within the scaffold, and migrated into cardiac muscles.

FIG. 34 provides high-magnification microscopic images of cardiac tissues showing locations of the transplanted cardiac stem cells in the myocardium layer of the myocardium infarcted rats 14 days after transplantation thereinto, wherein the uppermost panels represent the attached scaffold while the more lower images are responsible for more inner myocardia with DiI-labeled cardiac stem cells in red fluorescence (a), DAPI-stained nuclei (b), and merged images (c) indicative of the location of the DiI-labeled stem cells inside the myocardium layer and at the myocardium infarcted zone lacking myocardiocytes, demonstrating the migration of the transplanted cells into cardiac tissues therearound.

(11) Analysis for Improvement in Heart Function by Transplantation of Stem Cell-Loaded Porous 3D Scaffold into Disease Model 2

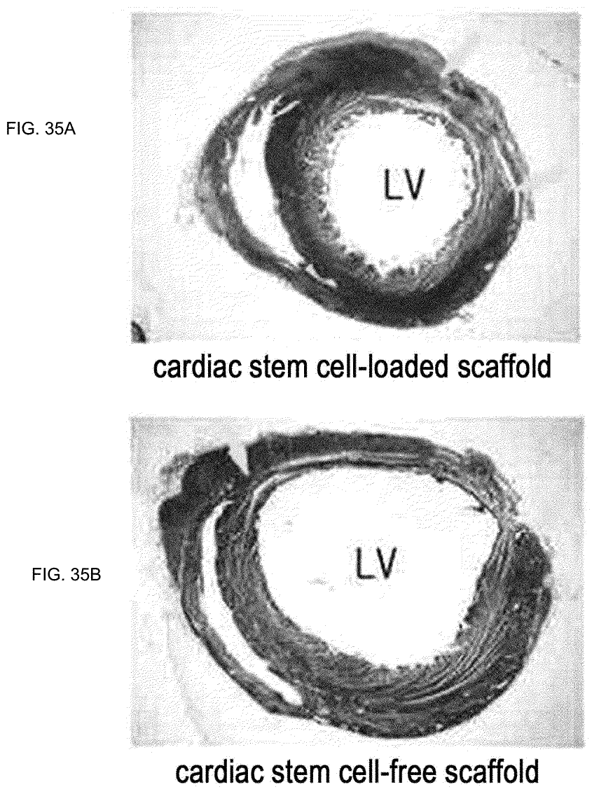

(A) Tissues obtained from the animal model of (10)-2) were paraffin sectioned and stained with H&E (hematoxylin and eosin) (FIG. 35). At 28 days of coronary artery ligation, cardiac infarction was successfully induced, and was also observed to cause myocardium loss and cardiomegaly.

As can be seen in FIG. 35, the cardiac stem cell-loaded scaffold of Example 7-(1) retained its morphology well, and made the LV anterior wall thicker than did the cell-free scaffold of Comparative Example 1. After being transplanted with the scaffold of Example 7-(1), the heart was less prone to LV dilatation, compared to the scaffold of Comparative Example 1, and allowed for the remarkable regeneration of cardiac muscles (as indicated by white arrows in FIG. 35).

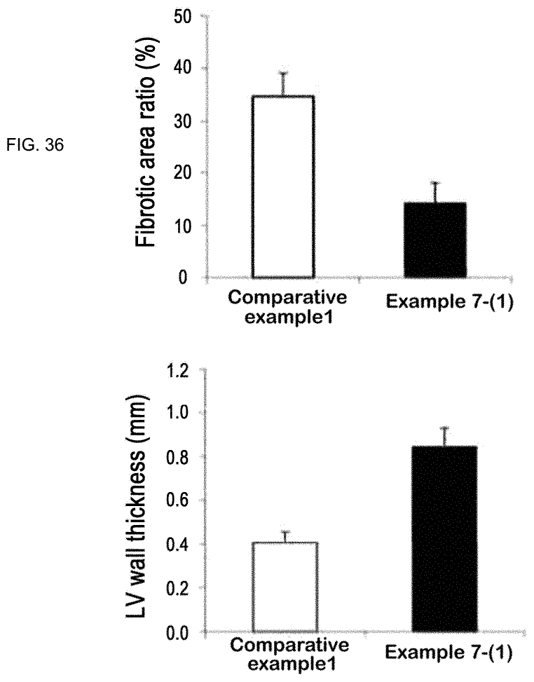

(B) On day 28 after the scaffolds of Example 7-(1) and Comparative Example 1 were transplanted as described in (A), the transplanted tissues were measured for fibrotic area and thickness. A significant reduction in fibrotic area was observed when the cardiac stem cell-loaded scaffold of Example 7-(1) was transplanted, compared to the cell-free scaffold of Comparative Example 1 (FIG. 36a). In addition, the cardiac stem cell-loaded scaffold of Example 7-(1) made the LV anterior wall thicker than did the cell-free scaffold of Comparative Example 1 (FIG. 36b).

(12) Effect of Thickness and Density of Scaffold on Cell Migration

The effect on myocardium regeneration of the thickness and density of the oriented scaffold of Example 7-(2) was examined.

The density of the scaffold of Example 7-(2) was five-fold reduced by applying physical force thereto. Briefly, the density was reduced from 14.5 g/cm³ to 2.7 g/cm³. Cell migration was examined in the scaffolds of Example 7-(2) before and after its density was reduced.

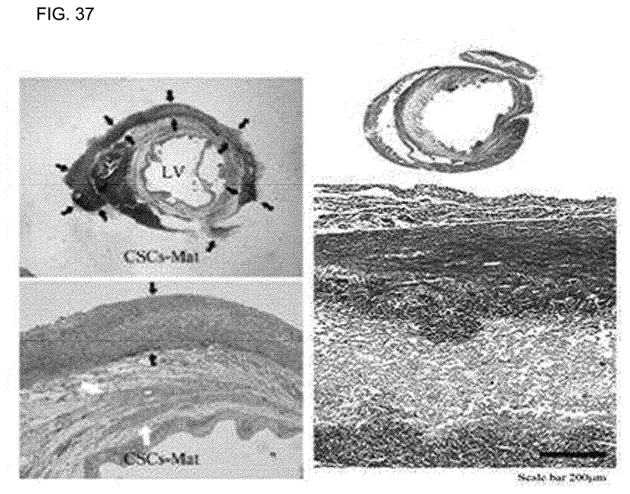

The stem cell-loaded scaffolds were transplanted into the damaged heart of the animal model of (10). On day 14 after transplantation, cardiac tissues were investigated. There was an increase in the migration and penetration of cells into the scaffold which swelled in volume and pores between entangled fibers as one or more physical forces were applied in opposite directions thereto (FIG. 37). However, the stem cells could not migrate into the scaffold which swelled too much due to excessive physical expansion. When the microfibrous mat was swelled too much by excessive physical force, the cells did not migrate into the scaffold and the tissue as indicated by white portions (right panel, FIG. 37).

Accordingly, it is required that the scaffold be allowed to swell to a proper thickness and volume.

(13) Effect of Grow Factor-Loaded Scaffold on Myocardial Regeneration and Angiogenesis

An examination was made of the ability of the growth factor-loaded fibrous porous scaffold of Example 7-(3) to regenerate myocadia. Briefly, infarcted myocardial zones transplanted with a fibrous porous 3D scaffold alone as a control, with a growth factor-loaded fibrous porous 3D scaffold, and with a stem cell/growth factor-loaded fibrous porous 3D scaffold were histologically stained to examine myocardial regeneration and angiogenesis.

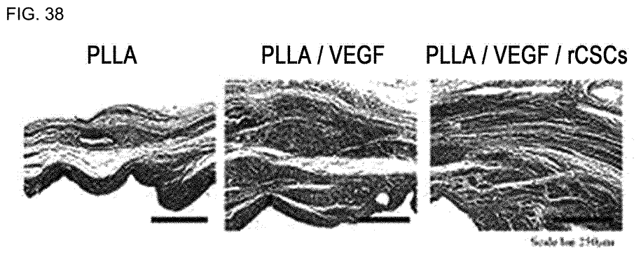

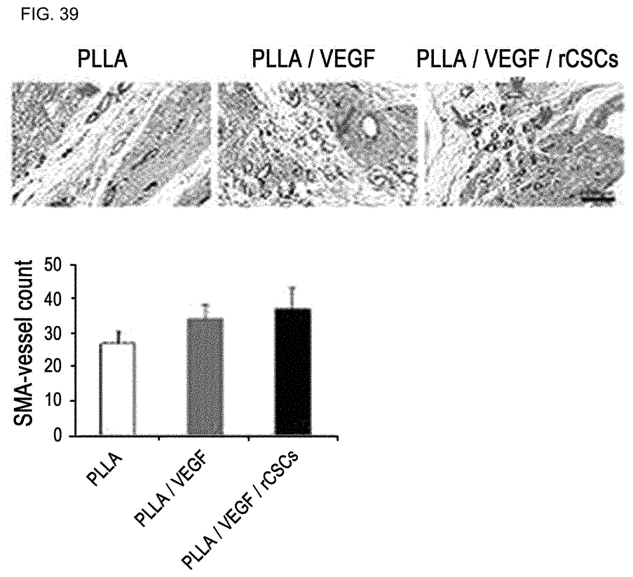

Compared to the scaffold alone, the VEGF-loaded scaffold and the stem cell/VEGF-loaded scaffold were found to improve myocardial regeneration (FIG. 38) and angiogenesis (FIG. 39), with the superiority of the stem cell/VEGF-loaded scaffold over the VEGF-loaded scaffold.

(14) Effect of Grow Factor-Loaded, Oriented Porous 3D Scaffold on Myocardial Regeneration and Angiogenesis

1) Morphological Observation and Proliferation of Cardiac Stem Cells Loaded on Scaffold

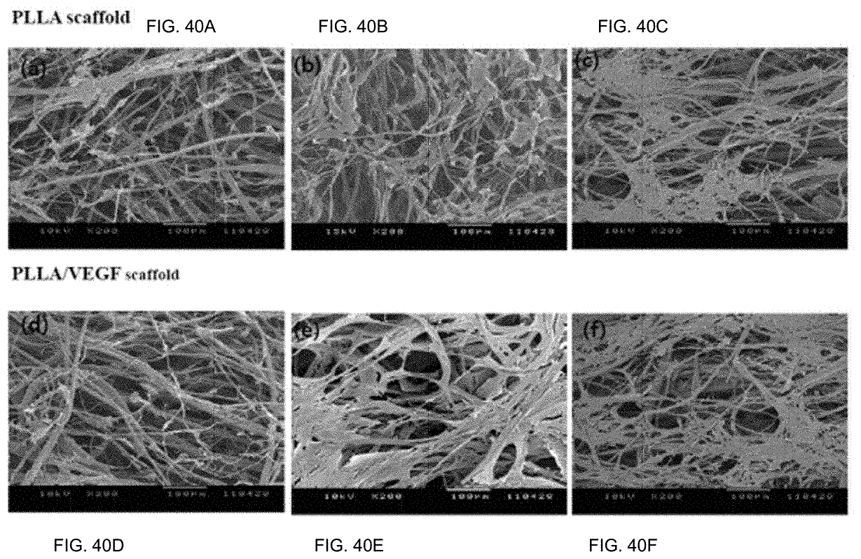

Before and after being loaded with VEGF, the 3D scaffold of Example 7-(2) was incubated for 7 days in a growth medium during which the scaffold was morphologically observed on day 1, day 5 and day 7. The results are depicted in FIG. 40.

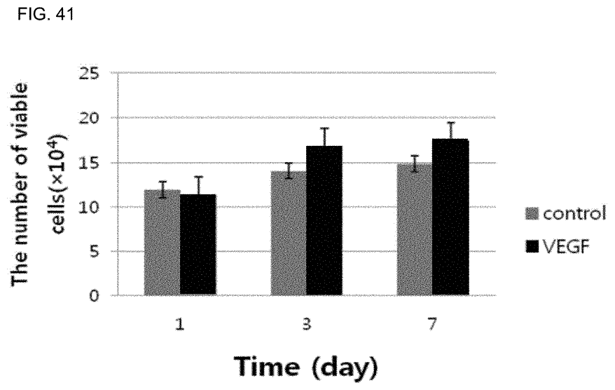

In addition, cells were counted using CCK-8, and the results are depicted in FIG. 41.

As can be seen in FIG. 41, the VEGF-loaded scaffold allowed for higher adhesion and proliferation of the cardiac stem cells.

2) Analysis for Improvement in Heart Function by Transplantation of Growth Factor- and/or Stem Cell-Loaded Porous 3D Scaffold into Disease Model

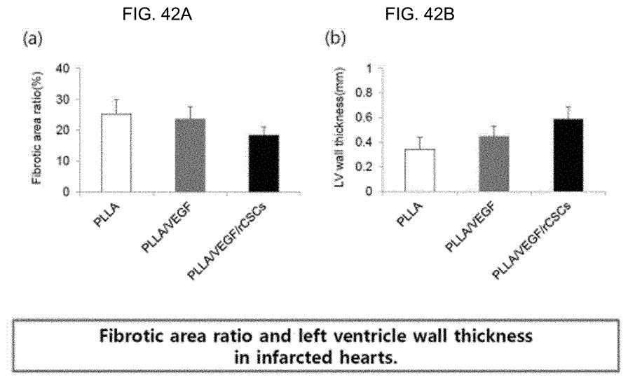

On day 28 after the scaffolds were transplanted into the animal model of (10), the transplanted tissues were measured for fibrotic area and thickness. Briefly, infarcted myocardial zones transplanted with fibrous porous 3D scaffolds of Example 7-(2) which were loaded with neither a growth factor nor stem cells (PLLA), with VEGF (PLLA/VEGF), and both VEGF and cardiac stem cells (PLLA/VEGF/rCSCs) were examined for fibrotic area and LV wall thickness. The results are given in FIG. 42.

As understood from the data of FIG. 42, both the PLLA/VEGF group and the PLLA/VEGF/rCSCs group decreased in fibrotic area and increased in LV anterior wall thickness, compared to the control. Particularly, for the scaffold loaded with both VEGF and cardiac stem cells, it reduced the fibrotic area by 10% and increased the LV anterior wall thickness about 1.5 times, compared to the control.

3) Echocardiographic Examination for Functional Recovery of the Heart

Groups transplanted with the scaffold of Example 7-(2) loaded with neither a growth factor nor stem cells (PLLA), the 3D scaffold of Example 7-(2) loaded with VEGF but not with stem cells (PLLA/VEGF), and the scaffold of Example 7-(2) loaded with both VEGF and stem cells (PLLA/VEGF/rCSCs) were examined for ejection fraction (EF) and fractional shortening (FS) to determine whether the groups transplanted with the scaffolds were improved in cardiac function.

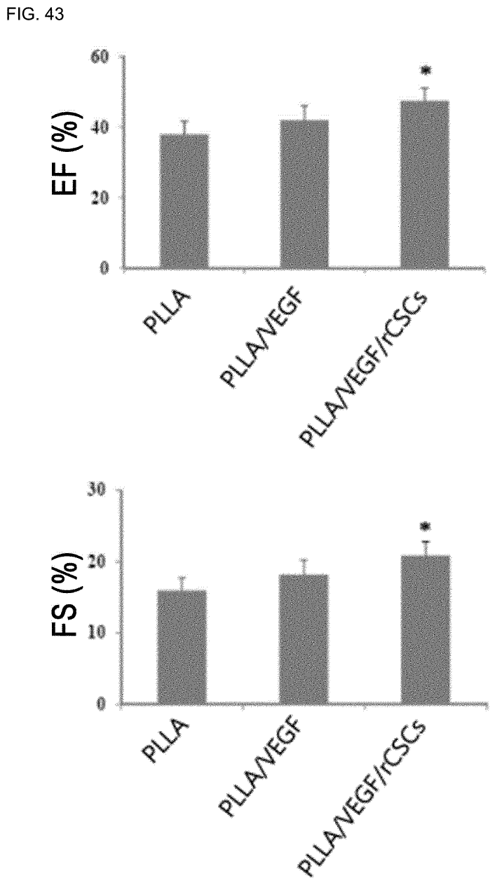

As shown in FIG. 43, the PLLA/VEGF/rCSCs group exhibited an EF value near the normal range (56-78%) and an FS value near the normal range (25-40%), too.

(15) Viability and Retention of Transplanted Cells

Animal models with a myocardium defect to which cardiac stem cells were directly injected, or a cardiac stem cell-loaded fibrin gel or a cardiac stem cell-loaded fibrous porous 3D scaffold was transplanted were examined for the viability and the retention at detective myocardial tissues of the cardiac stem cells.

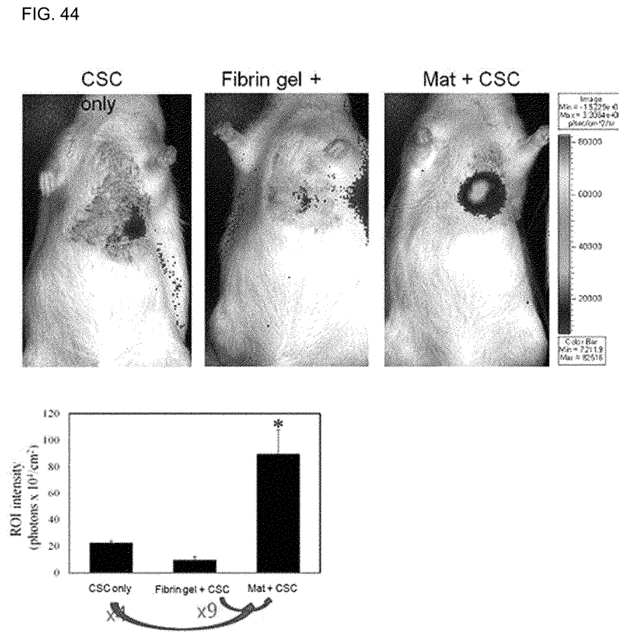

The transplantation of the cardiac stem cell-loaded scaffold of the present invention was observed to increase in cell viability and retention about four times compared to the direct injection of stem cells, and about nine times compared to the transplantation of the stem cell-loaded fibrin gel (FIG. 44). From these data, it is understood that the cell-loaded scaffold for tissue regeneration promises high viability and retention to the cells and allows for the effective delivery of the cells to a defective tissue, thereby bringing about a better tissue regeneration effect compared to conventional tissue regeneration methods.

(16) Release Control of VEGF Gene-Loaded Scaffold

1) Morphological Observation of Scaffold Loaded with pDNA Complex for Transfection

To see whether the scaffold of Example 7-(4) was stably loaded with a pDNA complex for transfection, SEM and fluorescence microscopy were used.

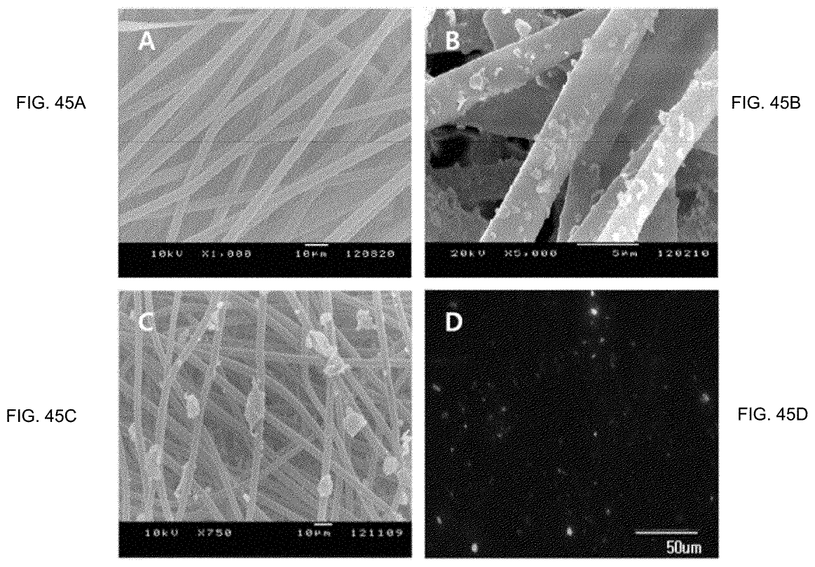

The results are given in FIG. 45. As can be seen in FIG. 45, unlike a scaffold free of a DNA complex (A), the fibrous porous 3D scaffold of Example 7-(4) was observed to be stably coated with a pDNA complex (B).

In addition, the following experiment was performed to examine whether the pDNA complex loaded to the scaffold was transfected into and expressed in cells. Briefly, the expression of the hVEGF165 protein was investigated by ELISA (enzyme-linked immunosorbent assay). With regard to the transfection of the pVEGF complex, rat cardiac stem cells were seeded at a density of 5×10⁵ cells/scaffold onto the pVEGF complex-loaded scaffolds which were then incubated for 2 hrs to induce the cells to adhere thereto. Then, all of the scaffolds were examined for the release of the gene for 14 days during which the medium was changed with a fresh one every two days. The cell culture media were taken on day 2, day 7 and day 14, and stored at −70° C. until use.

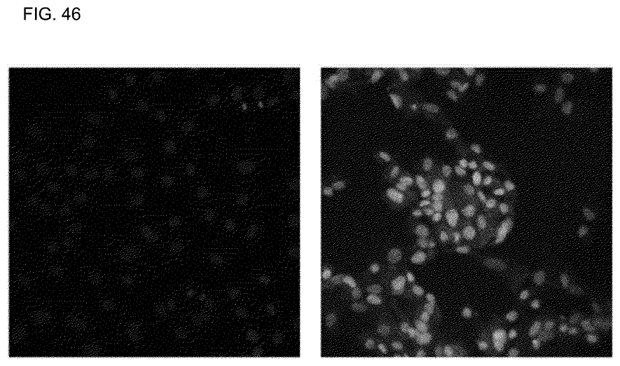

Fluorescence microscopy showed that the hVEGF165 was stably transfected into the cardiac stem cells and was expressed (right panel in FIG. 46).

2) Release Profile

To draw a pVEGF release profile of the scaffold, 3 μg of the pVEGF-loaded scaffold was incubated in 1 mL of PBS (pH 7.4) at 37° C. for 14 days. All samples were stirred at a constant speed (30 rpm). The release medium was changed with a fresh one freshly at determined times (1 hr, 6 hrs, 1 day, 2 days, 5 days, 7 days, 10 days and 14 days after the start of incubation). The withdrawn media were centrifuged, and used for the quantitative analysis of the pDNA released from the scaffold with the aid of the fluorescent dye PicoGreen (invitrogen, Eugene, Oreg., USA). The quantity of released pDNA was expressed as a mass percentage of released pDNA to the total mass of the loaded pDNA.

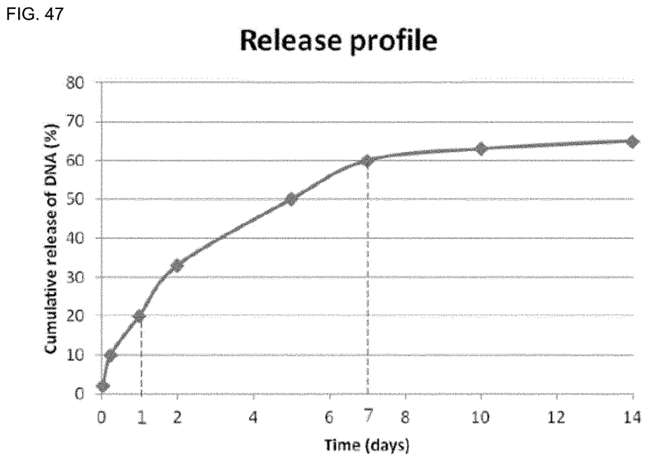

The result is depicted in FIG. 47. As can be seen, the release of DNA from the scaffold was initially 20% on day 1, and the increased to 60% over one week, and finally up to approximately 65% on day 14. These data implies that a gene can be locally released in a controlled manner through the scaffold of the present invention.

3) Sustenance of VEGF Expression in Transfected Cardiac Stem Cells

In regard to examining whether the oriented PLLA/PLGA scaffold which was coated with pVEGF and loaded with cardiac stem cells (referred to as “sustained release group”) allows for the sustained expression of hVEGF protein, the following experiment was conducted.

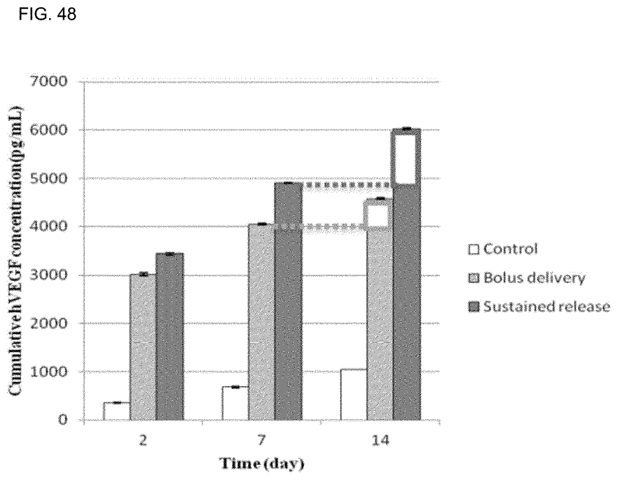

Briefly, a comparison was made of the expression of hVEGF protein between the sustained release group, and the group in which the oriented PLLA/PLGA scaffold was loaded with pVEGF-transfected cardiac stem cells (hereinafter referred to as “Bolus delivery”). For this, the sustained release group or a pVEGF complex was introduced in an equal amount into rat cardiac stem cells, followed by an ELISA analysis for determining hVEGF expression levels. The results are depicted in FIG. 48 and summarized in Table 1.

TABLE 1

| | Unit (pg/mL) | | |
|---|---|---|---|
| | Day 0-2 | Day 2-7 | Day 7-14 |
| Control | 356.25 | 325.25 | 358 |
| Bolus | 2901 | 1000 | 598 |
| Sustained | 3444.75 | 1456 | 1128.25 |

As is apparent from the data of FIG. 48 and Table 1, the hVEGF protein expressed from the cells loaded to the scaffold of the sustain release group was sustained at high levels over 14 days. In contrast, the bolus delivery group expressed hVEGF at low levels, compared to the sustained release group. Taken together, the results obtained above implied that the fibrous porous scaffold of the present invention, when loaded with a gene, such as pVEGF complex, allows for prolonged transfection of the gene, and enables the transfectant to express the target protein in a sustained manner.

1. A fluffy 3-dimensional (3D) porous scaffold comprising biodegradable polymer fibers, wherein the biodegradable polymer fibers in the scaffold are separably entangled with each other to form a 3D network structure.

2. The scaffold of claim 1, wherein the fiber is 1-15 μm in diameter.

3. The scaffold of claim 1, wherein the pore of the scaffold is 50 to 400 μm in diameter.

4. The scaffold of claim 1, wherein the porosity of the scaffold is 50 to 99%.

5. The scaffold of claim 1, wherein the thickness of the scaffold is 50 μm to 1.5 cm.

6. The scaffold of claim 1, wherein the scaffold can be attached directly to a target tissue.

7. The scaffold of claim 1, wherein the scaffold can alternatively expand or shrink in one-, two- or three-dimensional patterns in response to the swelling or contraction of the tissues to which the scaffold is attached.

8. The scaffold of claim 1, wherein the biodegradable polymer is one or more polymers selected from a group consisting of polylactic acid (PLA), polyglycolic acid (PGA), poly(D,L-lactide-co-glycolide) (PLGA), poly(caprolactone), diol/diacid aliphatic polyester, polyester-amide/polyester-urethane, poly(valerolactone), poly(hydroxyl butyrate) and poly(hydroxyl valerate).

9. The scaffold of claim 8, wherein the polymer is poly-L-lactic acid (PLLA).

10. The scaffold of claim 1, which further comprises a cell, drug or a combination thereof.

11. A method for preparing the scaffold of claim 1, comprising:
(i) preparing a spinning solution by dissolving biodegradable polymers in an organic solvent;
(ii) spinning the spinning solution by using an electro-spinner and volatilizing the organic solvent at the same time to form a microfibrous mat comprising biodegradable polymer fibers, which are separably entangled with each other in a network structure; and
(iii) expanding the microfibrous mat mechanically to form the fluffy 3D porous scaffold.

12. The method of claim 11, wherein the biodegradable polymer is one or more polymers selected from a group consisting of polylactic acid (PLA), polyglycolic acid (PGA), poly(D,L-lactide-co-glycolide) (PLGA), poly(caprolactone), diol/diacid aliphatic polyester, polyester-amide/polyester-urethane, poly(valerolactone), poly(hydroxyl butyrate) and poly(hydroxyl valerate).

13. The method of claim 11, wherein the fiber in step (ii) has 1 to 15 μm in a diameter.

14. The method of claim 11, wherein the organic solvent is selected from a group consisting of chloroform, dichloromethane, dimethylformamide, dioxane, acetone, tetrahydrofuran, trifluoroethane, hexafluoroisopropylpropanol (HFIP), dichloromethane/HFIP or dichloromethane/acetone.

15. The method of claim 11, wherein the organic solvent has a boiling point of 0-40° C. and a viscosity of 25-35 cps.

16. The method of claim 11, wherein the step (ii) is carried out under the following conditions; temperature: 15-25° C., humidity: 10-40%, spun distance: 10-20 cm, voltage: 10-20 kV, release speed: 0.050-0.150 ml/min and the internal diameter of the syringe: 0.5-1.2 mm.

17. An implantation material for tissue regeneration comprising the scaffold of claim 1.

18. The implantation material of claim 17, wherein the scaffold comprises a cell.
